(19) United States
(12) Patent Application Publication
(10) Pub. No.: US 2026/0168993 A1
(43) Pub. Date: Jun. 18, 2026

(54) ANALYTE BINDING COMPOSITIONS, METHODS, AND SYSTEMS

(71) Applicant: Freenome Holdings, Inc., Brisbane, CA (US)

(72) Inventors: Preston WILLIAMS, San Mateo, CA (US); Preetham PEDDIREDDY, Burlingame, CA (US); Phwe Phwe HLAING, Daly City, CA (US); Shilpa CHOUDHURY, Daly City, CA (US); Jared DEYARMIN, San Bruno, CA (US); Kevin DAI, San Francisco, CA (US); Danica-Mae MANALO, Burlingame, CA (US); Swaralee KULKARNI, San Mateo, CA (US)

(21) Appl. No.: 19/382,996

(22) Filed: Nov. 7, 2025

Related U.S. Application Data
(63) Continuation of application No. PCT/US2025/054388, filed on Nov. 6, 2025.
(60) Provisional application No. 63/717,700, filed on Nov. 7, 2024.

(51) Int. Cl. G01N 33/543; G01N 33/531

(52) U.S. Cl. CPC G01N 33/54326; G01N 33/531

(57) ABSTRACT

Described herein are analyte-binding compositions, methods of analysis, and analytical systems utilizing such compositions.

### CROSS-REFERENCE

This application is a continuation of International Application No. PCT/US2025/054388, filed Nov. 6, 2025, which claims the benefit of U.S. Provisional Application No. 63/717,700, filed Nov. 7, 2024, which is incorporated by reference herein in its entirety.

### BACKGROUND

A method of analyzing for certain substances or analytes may involve the use of a solid phase such as a functionalized bead which may selectively bind to a target substance or analyte, such as a protein, peptide, biomarker, and/or antibody.

### SUMMARY

In some assays, a solid phase may on its surface carry and/or display specific binding molecules which specifically bind the analyte. In order to improve detection and/or quantification of such analytes, improved and/or more uniform analyte-binding compositions and/or functionalized beads are needed.

In an aspect, the present disclosure provides a method of preparing a functionalized composition, the method comprising: (a) activating an analyte-binding composition comprising a plurality of functionalization sites at a surface; (b) reacting the analyte-binding composition with a first functionalization agent, wherein the first functionalization agent is amine modified, and wherein a first linker is interspaced between the amine and the first functionalization agent; and (c) linking a modified analyte to the first functionalization agent.

In some embodiments, the surface comprises a bead. In some embodiments, the bead comprises a carboxylated magnetic bead. In some embodiments, the surface comprises a planar surface. In some embodiments, the first functionalization agent comprises dibenzocyclooctyne (DBCO). In some embodiments, the first functionalization agent comprises an alkyne, a linear alkyne, an azide, a tetrazine, an alkene, a ring-strained alkyne, a terminal thiol, a thiol reactive agent, or a combination thereof. In some embodiments, the first linker comprises an alkyl, a polyglycerol, a polysarcosine, an amino acid spacer, a triazole, a carbamate, a carbonate, a zwitterionic spacer, or a combination thereof. In some embodiments, the first linker comprises PEG-2, PEG-4, PEG-8, PEG-12, PEG-16, PEG-20, PEG-24, or PEG-28. In some embodiments, the first linker comprises PEG-4. In some embodiments, the linking is performed using one or more click-chemistry reactions. In some embodiments, the modified analyte comprises an alkyne, a linear alkyne, an azide, a tetrazine, an alkene, a cycloalkyne, a terminal thiol, a thiol reactive agent, a maleimide haloacetyl, a pyridyl disulfide, a vinyl sulfone, an epoxide, or a combination thereof.

In some embodiments, the modified analyte comprises an azide modified analyte. In some embodiments, the azide in the azide modified analyte reacts with the DBCO of the first functionalization agent via a second linker. In some embodiments, the second linker comprises a polyethylene glycol (PEG). In some embodiments, the PEG is a PEG-N comprising PEG-1, PEG-2, PEG-3, PEG-4, PEG-5, PEG-6, PEG-7, PEG-8, PEG-9, or PEG-10. In some embodiments, N is more than or equal to about: 1, 2, 3, 4, 5, 6, 7, 8, 9, 10, 20, 30, 40, 50, 100, 200, 300, 400, or 500. In some embodiments, N is less than or equal to about: 1, 2, 3, 4, 5, 6, 7, 8, 9, 10, 20, 30, 40, 50, 100, 200, 300, 400, or 500. In some embodiments, the method comprises modifying the first linker, the second linker, or both. In some embodiments, the modifying promotes binding of the first linker, the second linker, or both to the surface or a target analyte. In some embodiments, the modifying of the first linker, the second linker, or both, is performed at least in part to optimize a separation distance between the amine and the first functionalization agent. In some embodiments, the first functionalization agent comprises DBCO. In some embodiments, the first or the second modified linker comprises PEG-1, PEG-2, PEG-3, PEG-4, PEG-5, PEG-6, PEG-7, PEG-8, PEG-9, or PEG-10. In some embodiments, the modifying of the first linker, the second linker, or both, is used to direct an orientation of the first functionalization agent with respect to the plurality of functionalization sites. In some embodiments, the first functionalization agent is dispersed more uniformly across the plurality of functionalization sites as compared to a functionalized composition prepared using a linker that is unmodified.

In some embodiments, the modified analyte comprises a target binding moiety. In some embodiments, the target binding moiety comprises a peptide. In some embodiments, the peptide comprises less than or equal to about 100 amino acids. In some embodiments, the peptide comprises more than or equal to 10 amino acids. In some embodiments, the peptide comprises about 20 to 40 amino acids. In some embodiments, the peptide comprises more than or equal to 40 amino acids. In some embodiments, the target binding moiety comprises an aptamer. In some embodiments, the aptamer comprises more than or equal to 30 bases. In some embodiments, the aptamer comprises less than or equal to about 100 bases. In some embodiments, the aptamer comprises about 40 to about 100 bases. In some embodiments, the aptamer comprises about 50 bases.

In some embodiments, the method comprises reacting the modified analyte with a second functionalization agent. In some embodiments, the second functionalization agent comprises a same chemical structure as the first functionalization agent. In some embodiments, the second functionalization agent comprises a different chemical structure as the first functionalization agent. In some embodiments, the method comprises, prior to b), determining a predicted orientation of the modified analyte, based at least in part on the first functionalization agent or the second functionalization agent. In some embodiments, the method comprises selecting the first functionalization agent or the second functionalization agent from a plurality of potential functionalization agents, based at least in part on the predicted orientation. In some embodiments, the second functionalization agent is amine modified, and wherein a third linker is interspaced between the amine and the second functionalization agent. In some embodiments, the third linker is longer than the first linker. In some embodiments, the third linker is shorter than the first linker. In some embodiments, the method comprises, prior to b), determining a predicted orientation of the modified analyte, based at least in part on a length of the first linker or a length of the third linker. In some embodiments, the method comprises selecting the length of the first linker or the length of the third linker from a plurality of potential lengths, based at least in part on the predicted orientation. In some embodiments, the third linker comprises a PEG-N comprising PEG-1, PEG-2, PEG-3, PEG-4, PEG-5, PEG-6, PEG-7, PEG-8, PEG-9, or PEG-10. In some embodiments, N is more than or equal to about: 1, 2, 3, 4, 5, 6, 7, 8, 9, 10, 20, 30, 40, 50, 100, 200, 300, 400, or 500. In some embodiments, N is less than or equal to about: 1, 2, 3, 4, 5, 6, 7, 8, 9, 10, 20, 30, 40, 50, 100, 200, 300, 400, or 500. In some embodiments, the method comprises linking the modified analyte to the second functionalization agent. In some embodiments, the modified analyte is linked to the first functionalization agent via a C-terminal and linked to the second functionalization agent via an N-terminal. In some embodiments, the modified analyte is modified at the N-terminal or the C-terminal.

In another aspect, the present disclosure provides a method of preparing a functionalized composition, the method comprising (a) activating an analyte-binding composition comprising a plurality of functionalization sites; (b) reacting the analyte-binding composition with a first functionalization agent, wherein the first functionalization agent is amine modified, and wherein a first linker is interspaced between the amine and the first functionalization agent; (c) reacting the analyte-binding composition with a second functionalization agent, wherein the second functionalization agent is amine modified, and wherein a second linker is interspaced between the amine and the second functionalization agent; and (d) coupling a modified analyte to the first functionalization agent at a first site and to the second functionalization agent at a second site.

In another aspect, the present disclosure provides an analyte-binding composition, comprising: (a) a surface comprising a plurality of functionalization sites; (b) one or more first functionalization agents linked via a first linker to at least one functionalization site of the plurality of functionalization sites; and (c) one or more second functionalization agents linked via a second linker to at least one first functionalization agent of the one or more first functionalization agents, wherein a functionalization site of the plurality of functionalization sites is uniformly dispersed from each other on the surface.

In another aspect, the present disclosure provides an analyte-binding composition, comprising: (a) a surface comprising a plurality of functionalization sites; (b) a first functionalization agent linked via a first linker to a first functionalization site of the plurality of functionalization sites; and (c) a second functionalization agent linked via a second linker to a second functionalization site of the plurality of functionalization sites, wherein the first functionalization site and the second functionalization site are separated by a uniform linear distance (d_{L}).

In some embodiments, di is more than or equal to about: 5 μm, 4 μm, 3 μm, 2 μm, 1 μm, 100 nm, 10 nm, 1 nm, or 0.1 nm. In some embodiments, di is less than or equal to about: 5 μm, 4 μm, 3 μm, 2 μm, 1 μm, 100 nm, 10 nm, 1 nm, or 0.1 nm. In some embodiments, di is configured for an analyte-binding region to link to the first functionalization agent at a first site, and link to the second functionalization agent at a second site. In some embodiments, the analyte-binding region comprises a target binding moiety. In some embodiments, the target binding moiety comprises a peptide. In some embodiments, the peptide comprises less than or equal to about 100 amino acids. In some embodiments, the peptide comprises more than or equal to 10 amino acids. In some embodiments, the peptide comprises about 20 to 40 amino acids. In some embodiments, the peptide comprises more than or equal to 40 amino acids. In some embodiments, the target binding moiety comprises an aptamer. In some embodiments, the aptamer comprises more than or equal to 30 bases. In some embodiments, the aptamer comprises less than or equal to about 100 bases. In some embodiments, the aptamer comprises about 40 to about 100 bases. In some embodiments, wherein the aptamer comprises about 50 bases.

In another aspect, the present disclosure provides an analyte-binding composition, comprising: a surface comprising a plurality of functionalization sites; one or more functionalization agents covalently linked to at least one functionalization site of the plurality of functionalization sites by one or more linking groups to yield a functionalized surface, wherein the covalently linked functionalization agents are distributed across the functionalized surface within one standard deviation of a uniform linear spacing distance (d_{L}) or within one standard deviation of a uniform radial spacing distance (d_{R}).

In some embodiments, the one or more linking groups comprise a bond. In some embodiments, the one or more linking groups comprise an amide, an amine, an ester, an alkyne/cycloalkyne or an azide. In some embodiments, the functionalized surface comprises a planar surface. In some embodiments, the functionalized surface comprises a surface of a bead. In some embodiments, d_{R} is less than or equal to about π/2. In some embodiments, d_{R} is less than or equal to about π/4. In some embodiments, a diameter of the bead is less than or equal to about 10 micrometers (μm). In some embodiments, the diameter of the bead is less than or equal to 1 μm. In some embodiments, the bead is a magnetic bead. In some embodiments, di is less than or equal to about: 5 μm, 4 μm, 3 μm, 2 μm, 1 μm, 100 nm, 10 nm, 1 nm, or 0.1 nm. In some embodiments, the one or more functionalization agents are covalently linked to more than or equal to about: 3, 5, 10, or 20 of the plurality of functionalization sites. In some embodiments, the analyte-binding composition is linked to a plurality of target analytes. In some embodiments, the analyte-binding composition is linked to about 10⁸ molecules or less per functionalized surface.

In some embodiments, the one or more functionalization agents comprise an amine, an azide, an alkyne, tetrazine or a cycloalkyne. In some embodiments, the amine comprises an amine modified cycloalkyne. In some embodiments, the amine comprises an amine modified cyclooctyne. In some embodiments, the amine modified cyclooctyne is a primary amine modified dibenzocyclooctyne (DBCO). In some embodiments, the amine modification comprises a polyethylene glycol linker. In some embodiments, the one or more functionalization agents comprises an alkyne, a linear alkyne, an azide, a tetrazine, an alkene, a cycloalkyne, a terminal thiol, a thiol reactive agent, or a combination thereof. In some embodiments, the polyethylene glycol linker is PEG-N, wherein Nis 2 or more, 3 or more, 4 or more, 5 or more, 6 or more, 7 or more, 8 or more, 9 or more, or 10 or more. In some embodiments, N is more than or equal to about: 1, 2, 3, 4, 5, 6, 7, 8, 9, 10, 20, 30, 40, 50, 100. In some embodiments, N is less than or equal to about: 1, 2, 3, 4, 5, 6, 7, 8, 9, 10, 20, 30, 40, 50, 100, 200, 300, 400, or 500. In some embodiments, the one or more functionalization agents comprise an analyte-binding region. In some embodiments, the analyte-binding region comprises an alkyne, a linear alkyne, an azide, a tetrazine, an alkene, a cycloalkyne, a terminal thiol, a thiol reactive agent, a maleimide haloacetyl, a pyridyl disulfide, a vinyl sulfone, an epoxide, or a combination thereof. In some embodiments, the analyte-binding region comprises an azide.

In some embodiments, the analyte-binding region comprises a target-binding moiety. In some embodiments, the target binding moiety comprises a peptide. In some embodiments, the peptide comprises less than or equal to about 100 amino acids. In some embodiments, the peptide comprises more than or equal to 10 amino acids. In some embodiments, the peptide comprises about 20 to 40 amino acids. In some embodiments, the peptide comprises more than or equal to 40 amino acids. In some embodiments, the target binding moiety comprises an aptamer. In some embodiments, the aptamer comprises more than or equal to 30 bases. In some embodiments, the aptamer comprises less than or equal to about 100 bases. In some embodiments, the aptamer comprises about 40 to about 100 bases. In some embodiments, the aptamer comprises about 50 bases.

In some embodiments, the analyte-binding composition comprises one or more second functionalization agents linked to the analyte-binding region. In some embodiments, the one or more second functionalization agents comprise a same chemical structure as the one or more functionalization agents. In some embodiments, the one or more second functionalization agents comprise a different chemical structure as the one or more functionalization agents. In some embodiments, the one or more functionalization agents or the one or more second functionalization agents are selected from a plurality of potential functionalization agents, based at least in part on a predicted orientation of the analyte-binding region. In some embodiments, the one or more second functionalization agents are linked to at least one functionalization site of the plurality of functionalization sites by one or more second linking groups. In some embodiments, the one or more second linking groups are longer than the one or more linking groups. In some embodiments, the one or more second linking groups are shorter than the one or more linking groups. In some embodiments, a length of the one or more linking groups or a length of the one or more second linking groups is selected from plurality of potential lengths, based at least in part on a predicted orientation of the analyte-binding region. In some embodiments, the one or more second linking groups comprises a PEG-N comprising PEG-1, PEG-2, PEG-3, PEG-4, PEG-5, PEG-6, PEG-7, PEG-8, PEG-9, or PEG-10. In some embodiments, N is more than or equal to about: 1, 2, 3, 4, 5, 6, 7, 8, 9, 10, 20, 30, 40, 50, 100, 200, 300, 400, or 500. In some embodiments, N is less than or equal to about: 1, 2, 3, 4, 5, 6, 7, 8, 9, 10, 20, 30, 40, 50, 100, 200, 300, 400, or 500. In some embodiments, the analyte-binding region is linked to the one or more functionalization agents via a C-terminal and linked to the one or more second functionalization agents via an N-terminal. In some embodiments, the analyte-binding region is modified at the N-terminal or the C-terminal.

In some embodiments, the one or more functionalization agents comprises PEG-4. In some embodiments, a total length of the one or more functionalization agents is configured to promote binding or interaction between the functionalized surface and a target analyte. In some embodiments, the target analyte comprises a protein, a peptide, a nucleotide, an antibody, or an aptamer. In some embodiments, the target analyte comprises the antibody. In some embodiments, the target analyte comprises the peptide. In some embodiments, the target analyte comprises the protein. In some embodiments, the one or more functionalization agents comprise one or more antigen-binding sites. In some embodiments, more than or equal to about: 30%, 50%, 80%, or 95% of the one or more antigen-binding sites are uniformly oriented toward an outer interface of the functionalized surface.

In some embodiments, the one or more functionalization agents comprise a linker which comprises a polyethylene glycol (PEG), an amine, a cycloalkyne, or a combination thereof. In some embodiments, the linker comprises an alkyl, a polyglycerol, a polysarcosine, an amino acid spacer, a triazole, a carbamate, a carbonate, a zwitterionic spacer, or a combination thereof. In some embodiments, the one or more functionalization agents comprise:

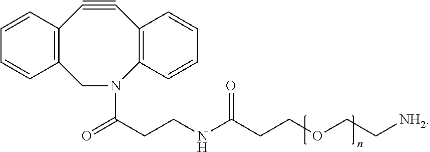

In some embodiments, n is 1 to 30. In some embodiments, n is 2, 4, 8, 12, 16, 24, or 28. In some embodiments, n is 4. In some embodiments, n is 8. In some embodiments, n is 12. In some embodiments, n is 23. In some embodiments, the analyte-binding composition comprises a fluorescent tag. In some embodiments, the fluorescent tag is covalently linked to the functionalized surface. In some embodiments, the fluorescent tag comprises fluorescein. In some embodiments, the covalently linked functionalization agents are distributed across the functionalized surface within one standard deviation of d_{L}. In some embodiments, the covalently linked functionalization agents are distributed across the functionalized surface within one standard deviation of d_{R}. In some embodiments, the functionalization agent is covalently linked to the plurality of carboxylated functionalization sites via ring-strained copper-free click chemistry.

In another aspect, the present disclosure provides a method of functionalizing a surface, the method comprising: (a) providing a surface comprising a plurality of carboxylated functionalization sites; (b) providing a functionalization agent comprising a primary amine and an analyte-binding region; and (c) covalently linking the functionalization agent to the plurality of carboxylated functional sites and/or to an analyte to obtain the functionalized surface.

In some embodiments, the functionalized surface comprises a planar surface. In some embodiments, the functionalized surface comprises a surface of a bead. In some embodiments, the analyte-binding region is configured to bind a target analyte. In some embodiments, the analyte-binding region comprises a target-binding moiety. In some embodiments, the target binding moiety comprises a peptide. In some embodiments, the peptide comprises less than or equal to about 100 amino acids. In some embodiments, the peptide comprises more than or equal to 10 amino acids. In some embodiments, the peptide comprises about 20 to 40 amino acids. In some embodiments, the peptide comprises more than or equal to 40 amino acids. In some embodiments, the target binding moiety comprises an aptamer. In some embodiments, the aptamer comprises more than or equal to 30 bases. In some embodiments, the aptamer comprises less than or equal to about 100 bases. In some embodiments, the aptamer comprises about 40 to about 100 bases. In some embodiments, the aptamer comprises about 50 bases. In some embodiments, the method comprises covalently linking a second functionalization agent to the plurality of carboxylated functionalization sites, wherein the second functionalization agent is linked to the analyte-binding region. In some embodiments, the second functionalization agent comprises a same chemical structure as the functionalization agent. In some embodiments, the second functionalization agent comprises a different chemical structure as the functionalization agent. In some embodiments, the method comprises, prior to b), determining a predicted orientation of the analyte-binding region, based at least in part on the functionalization agent or the second functionalization agent. In some embodiments, the method comprises selecting the functionalization agent or the second functionalization agent from a plurality of potential functionalization agents, based at least in part on the predicted orientation. In some embodiments, the analyte-binding region is linked to the functionalization agent via a C-terminal and linked to the second functionalization agent via an N-terminal. In some embodiments, the analyte-binding region is modified at the N-terminal or the C-terminal.

In another aspect, the present disclosure provides a method of measuring target binding, the method comprising: (a) providing a surface comprising a plurality of carboxylated functionalization sites; (b) providing one or more functionalization agents comprising a primary amine and an analyte-binding region; (c) covalently linking the one or more functionalization agents to the plurality of activated carboxylated functional sites to obtain a functionalized surface; (d) introducing the functionalized surface to a sample comprising a target analyte to promote binding between the analyte-binding region and the target analyte; and (e) measuring the binding between the functionalized surface and the target analyte.

In some embodiments, the functionalized surface comprises a planar surface. In some embodiments, the analyte-binding region comprises an alkyne, a linear alkyne, an azide, a tetrazine, an alkene, a cycloalkyne, a terminal thiol, a thiol reactive agent, a maleimide haloacetyl, a pyridyl disulfide, a vinyl sulfone, an epoxide, or a combination thereof. In some embodiments, the analyte-binding region comprises an azide. In some embodiments, the analyte-binding region comprises a target-binding moiety. In some embodiments, the target binding moiety comprises a peptide. In some embodiments, the peptide comprises less than or equal to about 100 amino acids. In some embodiments, the peptide comprises more than or equal to about 10 amino acids. In some embodiments, the peptide comprises about 20 to 40 amino acids. In some embodiments, the peptide comprises more than or equal to 40 amino acids. In some embodiments, the target binding moiety comprises an aptamer. In some embodiments, the aptamer comprises more than or equal to 30 bases. In some embodiments, the aptamer comprises less than or equal to about 100 bases. In some embodiments, the aptamer comprises about 40 to about 100 bases. In some embodiments, the aptamer comprises about 50 bases. In some embodiments, the target analyte comprises an antibody, an aptamer, a nucleotide, a protein, or a peptide.

In some embodiments, the method comprises covalently linking one or more second functionalization agents to the plurality of activated carboxylated functional sites, wherein the second functionalization agent is linked to the analyte-binding region. In some embodiments, the one or more second functionalization agents comprise a same chemical structure as the one or more functionalization agents. In some embodiments, the one or more second functionalization agents comprise a different chemical structure as the first functionalization agent. In some embodiments, the method comprises selecting the one or more functionalization agents or the one or more second functionalization agents from a plurality of potential functionalization agents, based at least in part on a predicted orientation of the analyte-binding region. In some embodiments, the analyte-binding region is linked to the one or more functionalization agents via a C-terminal and linked to the one or more second functionalization agents via an N-terminal. In some embodiments, the analyte-binding region is modified at the N-terminal or the C-terminal.

In some embodiments, the one or more functionalization agents are covalently linked through one or more linking groups. In some embodiments, the one or more linking groups comprise an alkyl, a polyglycerol, a polysarcosine, an amino acid spacer, a triazole, a carbamate, a carbonate, a zwitterionic spacer, or a combination thereof. In some embodiments, the one or more linking groups comprise a polyethylene glycol linker. In some embodiments, the polyethylene glycol linker is PEG-N, wherein Nis 2 or more, 3 or more, 4 or more, 5 or more, 6 or more, 7 or more, 8 or more, 9 or more, or 10 or more. In some embodiments, N is more than or equal to about: 1, 2, 3, 4, 5, 6, 7, 8, 9, 10, 20, 30, 40, 50, 100. In some embodiments, N is less than or equal to about: 1, 2, 3, 4, 5, 6, 7, 8, 9, 10, 20, 30, 40, 50, 100, 200, 300, 400, or 500.

In some embodiments, the covalently linked functionalization agents are distributed across the surface within one standard deviation of a uniform linear spacing distance (d_{L}) or within one standard deviation of a uniform radial spacing distance (d_{R}). In some embodiments, the one or more linking groups comprise a bond. In some embodiments, the one or more linking groups comprise an amide, an amine, an ester, or an azide. In some embodiments, the functionalized surface comprises a surface of a bead. In some embodiments, d_{R} is less than or equal to about π/2. In some embodiments, d_{R} is less than or equal to about π/4. In some embodiments, a diameter of the bead is less than or equal to about 10 μm. In some embodiments, the bead is a magnetic bead. In some embodiments, di is less than or equal to about: 5 μm, 4 μm, 3 μm, 2 μm, 1 μm, 100 nm, 10 nm, 1 nm, or 0.1 nm. In some embodiments, the one or more functionalization agents are covalently linked to more than or equal to about: 1, 3, 5, 10, or 20 of the plurality of functionalization sites.

In some embodiments, the one or more functionalization agents comprises an amine, an alkyne, a linear alkyne, an azide, a tetrazine, an alkene, a cycloalkyne, a terminal thiol, a thiol reactive agent, or a combination thereof. In some embodiments, the amine comprises an amine modified cyclooctyne. In some embodiments, the amine modified cyclooctyne is a primary amine modified dibenzocyclooctyne (DBCO). In some embodiments, the method comprises optimizing a total length of the one or more functionalization agents to promote binding or interaction between the functionalized surface and the target analyte. In some embodiments, the one or more functionalization agents comprise one or more antigen-binding sites. In some embodiments, more than or equal to about: 30%, 50%, 80%, or 95% of the one or more antigen-binding sites are oriented toward an outer interface of the functionalized surface. In some embodiments, the one or more functionalization agents comprise a polyethylene glycol (PEG). In some embodiments, the one or more functionalization agents comprise:

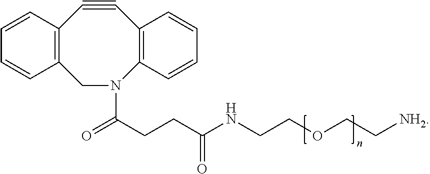

In some embodiments, n is 1 to 30. In some embodiments, n is 2, 4, 8, 12, 16, 24, or 28. In some embodiments, n is 4. In some embodiments, n is 8. In some embodiments, n is 12. In some embodiments, n is 23.

In some embodiments, the method comprises use of a fluorescent tag. In some embodiments, the fluorescent tag is covalently linked to the functionalized surface. In some embodiments, the fluorescent tag comprises fluorescein. In some embodiments, the functionalization agent is covalently linked to the plurality of carboxylated functionalization sites via ring-strained copper-free click chemistry. In some embodiments, (e) further comprises measuring the binding using a member selected from the group consisting of: a mass spectrometer, a fluorescence microscope, a flow cytometer, a fluorescence spectrophotometer, a surface plasmon resonance detector, or an enzyme-linked immunosorbent assay (ELISA) detector.

In another aspect, the present disclosure provides a system comprising (i) an optical detector, and (ii) any of the analyte-binding composition disclosed herein. In some embodiments, the optical detector is selected from the group consisting of: a fluorescence microscope, a flow cytometer, a fluorescence spectrophotometer, a surface plasmon resonance detector, or an enzyme-linked immunosorbent assay (ELISA) detector.

In another aspect, the present disclosure provides a non-transitory computer readable medium comprising machine executable code that, upon execution by one or more computer processors, implements any of the methods above or elsewhere herein.

In another aspect, the present disclosure provides a system comprising one or more computer processors and computer memory coupled thereto. The computer memory comprises machine executable code that, upon execution by the one or more computer processors, implements any of the methods above or elsewhere herein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the inventive concepts are set forth with particularity in the appended claims. A better understanding of the features and advantages of the inventive concepts will be obtained by reference to the following detailed description that sets forth illustrative embodiments, in which the principles of the inventive concepts are utilized, and the accompanying drawings (also “Figure” and “FIG.” herein), of which:

FIG. 1 illustrates a comparison of an example analyte-binding composition with improved uniformity (left) to an analyte-binding composition with less ideal uniformity (right).

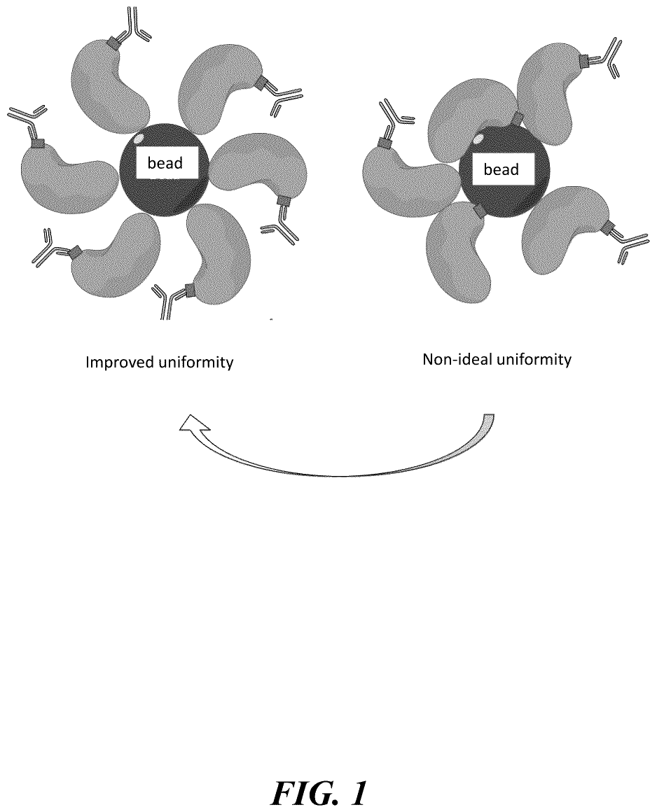

FIG. 2 illustrates an assay of an example of analyte-binding compositions.

FIG. 3 illustrates an example of a reaction scheme of a method for synthesis of analyte-binding compositions.

FIG. 4 illustrates an example of a reaction scheme of a method for synthesis of analyte-binding compositions.

FIG. 5 illustrates an example of a reaction scheme of a method for synthesis of analyte-binding compositions.

FIG. 6 illustrates an assay of analytical performance of an example analyte-binding composition.

FIG. 7 illustrates an assay of analytical performance of an analyte-binding composition.

FIG. 8 provides a table summarizing the results of the model system described herein.

FIG. 9 provides an embodiment of the model system described herein highlighting that the only time signal is observed is when all elements of the analyte-binding composition process described herein are present. A signal threshold of 104 was imposed and all measurement conditions that result in a signal below this threshold are considered to demonstrate no binding of the detection antibody.

FIG. 10 provides an example of a negative control experiment highlighting that each of the functional groups of the presently disclosed analyte-binding composition is required for signal detection.

FIG. 11 illustrates an example of plasma samples from 2 representative human subjects demonstrating significant differences in signals resulting from beads/surfaces coated with different peptide molecules derived from the same protein using the disclosed analyte-binding compositions.

FIG. 12 illustrates examples of functionalization agents comprising polyethylene glycol (PEG) linkers of various lengths.

FIG. 13 illustrates examples of target-binding moieties comprising peptides.

FIG. 14 illustrates an assay of analytical performance of example analyte-binding compositions having target-binding moieties comprising peptides.

FIG. 15 illustrates an assay of analytical performance of example analyte-binding compositions having target-binding moieties comprising peptides.

FIG. 16A illustrates an assay of analytical performance of example analyte-binding compositions.

FIG. 16B illustrates an assay of analytical performance of example analyte-binding compositions.

FIG. 17 illustrates an assay of analytical performance of an example analyte-binding composition.

FIG. 18 illustrates an assay of analytical performance of example analyte-binding compositions comprising various linker lengths.

FIG. 19 illustrates an example of an analyte-binding region modified at two sites.

FIG. 20A illustrates an example of an analyte-binding region linked to one functionalization agent.

FIG. 20B illustrates an example of an analyte-binding region linked to two functionalization agents.

FIG. 21A illustrates an example of an orientation of an analyte-binding region linked to two functionalization agents.

FIG. 21B illustrates an assay of analytical performance of an analyte-binding composition comprising an analyte-binding region linked to two functionalization agents.

FIG. 22 shows a computer system that is programmed or otherwise configured to implement methods provided herein.

### DETAILED DESCRIPTION

While various embodiments of the inventive concepts have been shown and described herein, it will be obvious to those skilled in the art that such embodiments are provided by way of example only. Numerous variations, changes, and substitutions may occur to those skilled in the art without departing from the inventive concepts. It should be understood that various alternatives to the embodiments of the inventive concepts described herein may be employed.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

Certain inventive embodiments herein contemplate numerical ranges. When ranges are present, the ranges include the range endpoints. Additionally, every sub range and value within the range is present as if explicitly written out. The term “about” or “approximately” may refer to within an acceptable error range for the particular value, which may depend in part on how the value is measured or determined, e.g., the limitations of the measurement system. For example, “about” may refer to within 1 or more than 1 standard deviation. Alternatively, “about” may refer to a range of up to 20%, up to 10%, up to 5%, or up to 1% of a given value. Where particular values are described in the application and claims, unless otherwise stated the term “about” generally refers to within an acceptable error range for the particular value.

Provided herein are methods for solid surface and bead preparation and/or functionalization for protein biomarker discovery/confirmation efforts. In some cases, beads may be Luminex beads, which are functionalized according to methods described herein.

Such beads may be used to measure proteins for a disease prescreening, detection, or diagnostic protein panel. In some cases, various antigens (proteins, antibodies, peptides and aptamers) are linked to the one or more beads by methods described herein. In some cases, protein measurements are performed using functionalized beads provided herein. In some cases, analyte-binding compositions are used to measure peptides and/or aptamers.

Methods described herein may utilize multiple chemical conjugation approaches (e.g., EDC/NHS cross-linking and click chemistry) to functionalize solid and bead surfaces (e.g., magnetic and agarose) with different antigens (e.g., proteins, peptides, antibodies and aptamers) for use in multiple downstream assays comprising the use of a solid surface and/or bead (e.g., Luminex® bead-based assays).

One specific example is use of carboxylated beads and/or surfaces. To complete this chemical coupling process, multiple chemistry reaction may be performed including:
1.) Activation of the carboxylated surface
2.) Linkage/Synthesis of chemical reactive handle on molecule of interest in the correct position (peptide, protein, aptamer, etc.)
3.) Linkage of the analyte of interest to the product from operation 2 above for subsequent use in an analytical measurement assay.

Two commonly encountered problems directly addressed by this disclosure are as follows: analyte availability for downstream analytical assay development through primary amine chemistry (located on the side chain of Lysine amino acids and the N-terminus of proteins) is often limited if there are no lysine amino acids and the N-terminus is unavailable for reaction since primary amine chemistry may fail to link the antigen to the surface or bead in such cases. However, if there are highly abundant primary amines (Lysines) in a protein target, there are many different locations where the chemical conjugation may occur, which result in many different orientations of the antigen on the surface or bead. This may lead to antigens (e.g., protein, antibody, peptide or aptamer) to be displayed on the surface in random orientations (because of the vast number of conjugation sites).

Such random analyte orientation may negatively impact assay performance, especially for antibodies. If a lysine amino acid is present in the antigen binding site, the antibodies which are conjugated through that binding region may not be active. Furthermore, proteins that are recognized by auto-antibodies may require specific regions to be displayed from the surfaces, again if the binding epitope region impaired, the assay may be negatively impacted.

Another problem addressed by this disclosure is the inability of primary amine chemistry to properly conjugate smaller peptides and alternative affinity reagents (e.g. aptamers). Smaller analytes, such as peptides, may be challenging to conjugated to surfaces, and may suffer from poor conjugation efficiency. As analytes decrease in size, orientation may also play a bigger role.

Approaches described herein may address these challenges by providing methods to conjugate any antigen/molecule to a bead or surface. This opens up the availability to conjugate antigens that are not commercially available. Further, methods described herein may control the orientation of all molecules onto the surface since the reaction is highly specific, leading to improved analyte-binding compositions. A user may add the reactive component to any position on the antigen and optimize the presentation for assay measurement, with some orientations resulting in better signal to noise and overall assay performance.

The approaches described in this disclosure may be amenable to synthetically generated analytes, which may include peptides and aptamers. In synthesis, the reaction moiety may be inserted into any position that the user requires at the time of synthesis. For biologically generated reagents (e.g., recombinant proteins), there may be an additional operation of labeling the molecule with the reagent prior to conjugation to a surface or bead. This labeling strategy on biologically generated molecules may reduce the orientation control or may require more advanced approaches prior to reagent generation.

Methods described herein may use primary amine modified cyclooctynes to functionalize beads and surfaces (e.g., Luminex beads) using reagents similar to those used in Proximity based degradation (PROTRAC). Primary amine modified DBCO reagents may have different spacer lengths between the amine (which reacts with the activated surface) and the DBCO (which is used for a subsequent operation to click on an analyte of interest). This spacer between these two reactive elements may be very important and may be optimized to a particular application, since the length between the two groups may be changed depending on the spacer used.

Another benefit of methods described herein may be that the orientation of the analyte on the bead/surface is highly controlled. Due to the specific nature of the click chemistry that is being utilized, the conjugation reaction may occur between the DBCO and the azide. In some workflows, such as a workflow which identifies new putative auto-antibody hits, the resulting analyte used in downstream assays may be a peptide that may be on the smaller end of size (<88 amino acids). Similarly, a discovery assay may be executed such that peptides are presented from a surface of a phage virus into solution—this peptide is attached to the phage virus by the N-terminus, therefore having a method to display the peptide in the same. This method may not only ensure that the peptides were conjugated through the N-terminus exclusively. Furthermore, the peptides (or other small molecules) may be tuned to the optimal presentation distance through the use of different linkers.

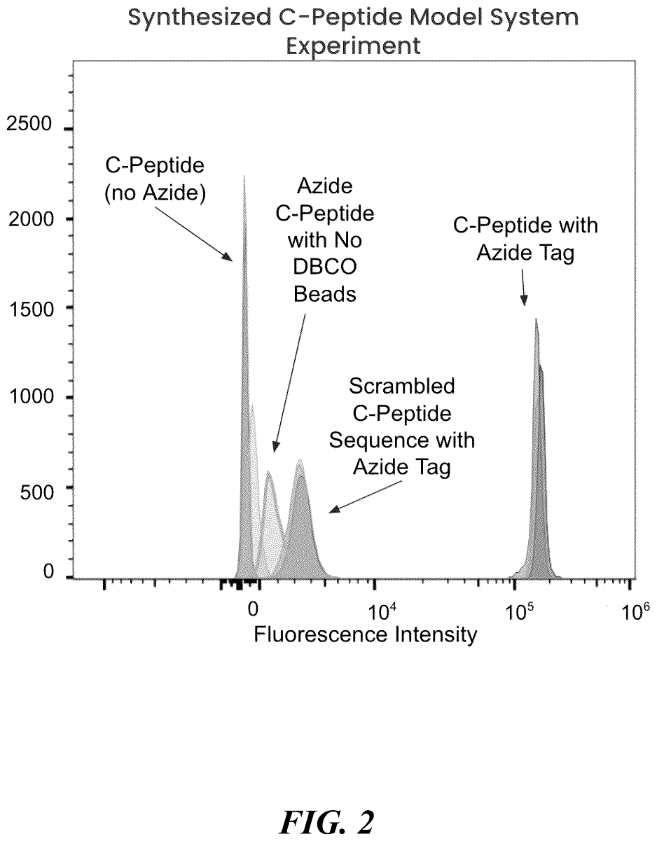

FIG. 2 illustrates an assay example (with a single linker length) assessed, which shows that each operation of the process is important for signal generation; if any part is removed, the signal decreases significantly.

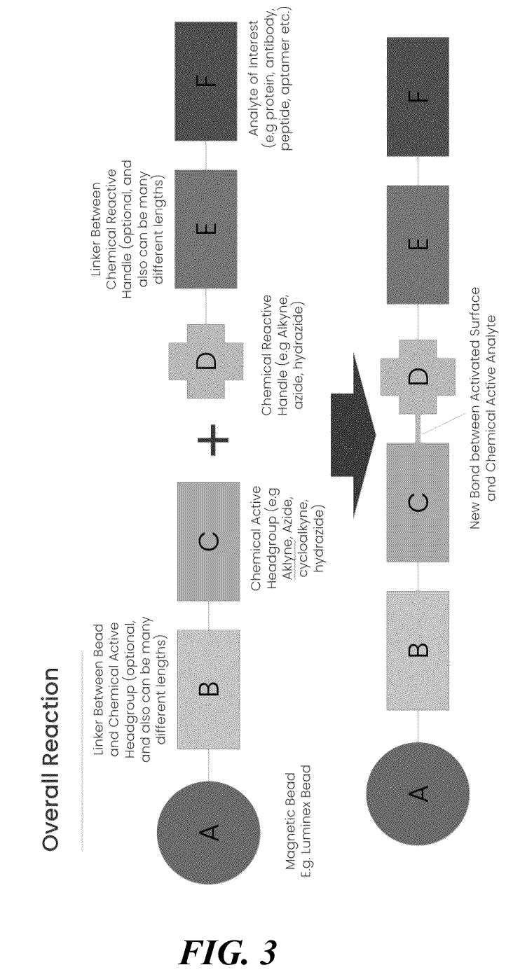

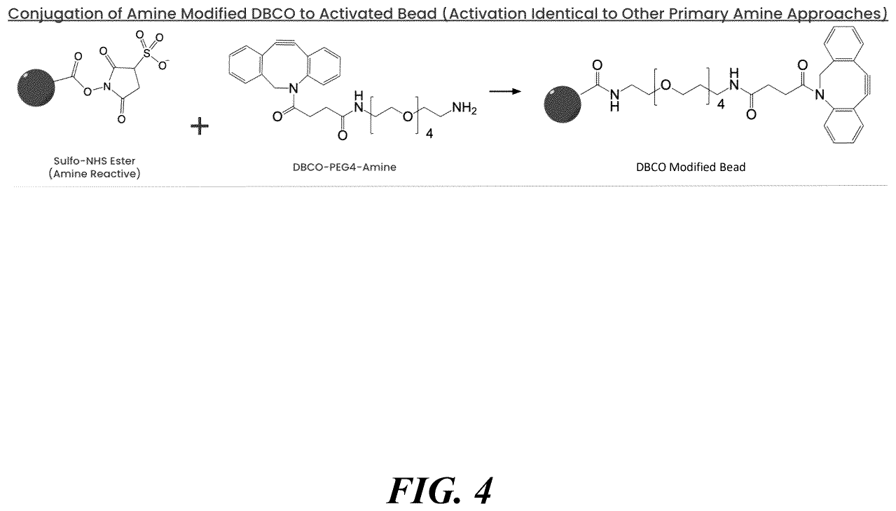

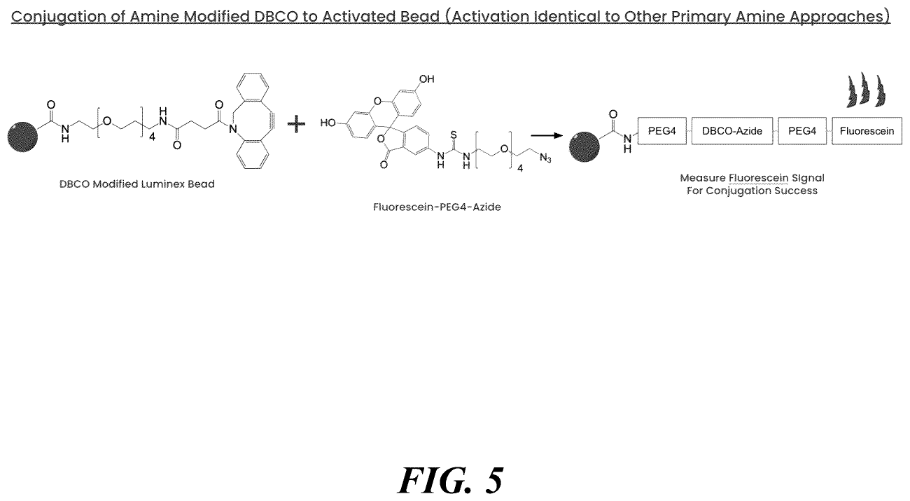

Another potential path is that the reaction may be flipped. In this case, the azide may be functionalized. Example workflows for functionalization processes described herein are illustrated in FIG. 3, FIG. 4, and FIG. 5.

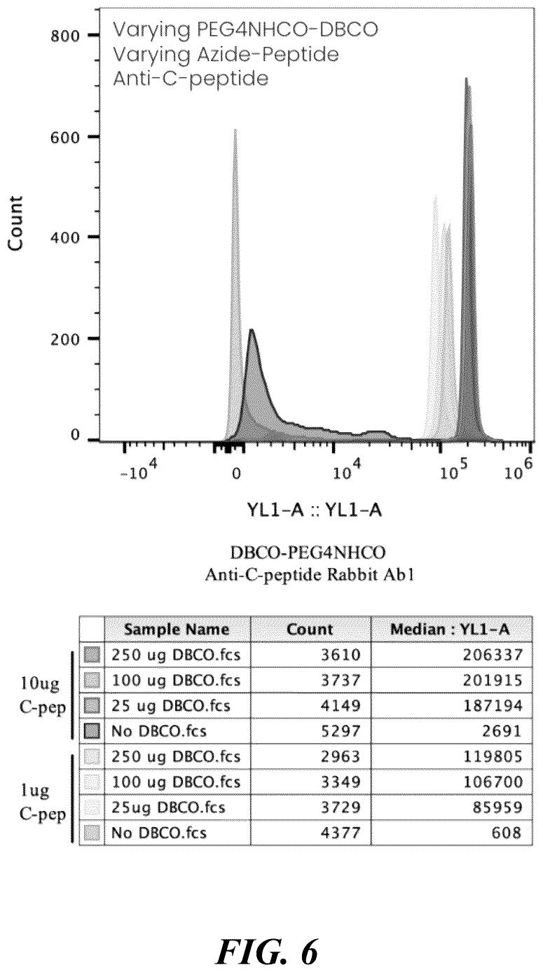

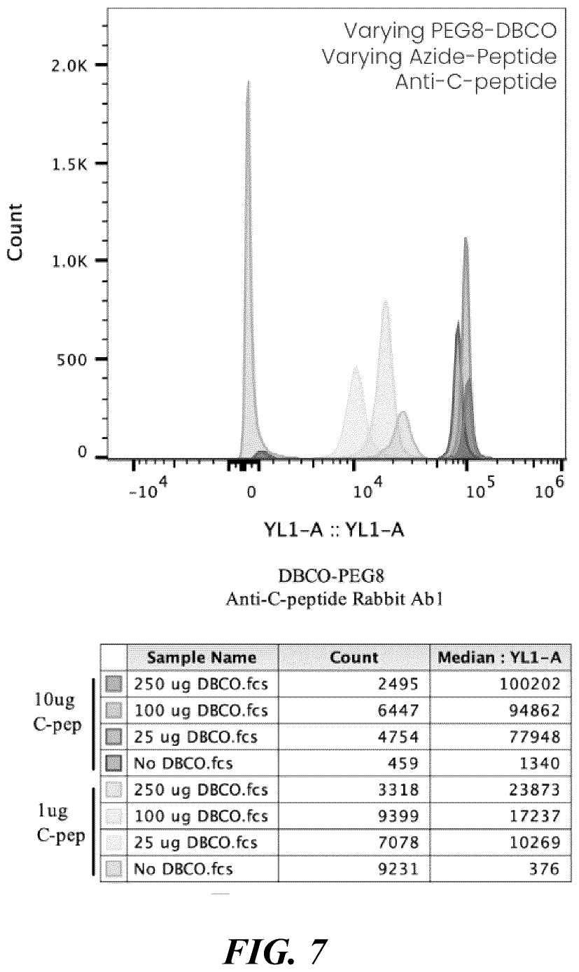

Two different linker lengths were examined in FIG. 6 and FIG. 7. FIG. 6 shows better performance through increased signal (on the X-axis) compared to the other linker length tested in FIG. 7, which showed lower signal overall.

Methods and compositions of this disclosure may be used for Luminex bead-based measurements, protein based measurements (e.g., using antibody coated beads and/or aptamer coated beads), antibody based measurements (e.g., using protein and/or peptide coated beads), protein class enrichment (e.g., by coating beads with different chemical functionalities), and/or combinations thereof.

In some cases, different classes may comprise different antigens, proteins, antibodies, peptides and aptamers, and or carboxylated surfaces. Linker length may be optimized according to the specific application and/or measurement.

The PEG linker may be any length, such as lengths between 2-20 PEG repeats. The method of the present disclosure may not only use chemistry not found in relation to carboxylated surfaces (Luminex Beads), but also identify the optimal length of the PEG linker. A too-long length may increase non-specific binding, while a too-short length may not display the antigen effectively for assay use. Below is a set of examples where the PEG Linker ranges from 4-23 units. These linkers may be important for antigen presentation in the analytical assay. Certain linkers may work well for some antigens while other linker lengths may work better for other antigens-so methods described herein may assess multiple linker lengths and find the optimal one for each antigen resulting in superior assay performance.

Highly specific “click chemistry” may be used for the chemical conjugation between the DBCO-modified surfaces and the analyte of interest. These analytes may span several different classes and include proteins, antibodies, peptides and aptamers. The analyte class may be required to have an azide modification present. This azide modification may be introduced through a number of ways, but an important aspect is that the azide modification is displayed at the correct length to facilitate the reaction with DBCO located on the bead surface. As above, the length may be varied and this method identifies the ideal linker length for successful conjugation between the DBCO-decorated beads and the azide modified analytes

### Methods for Preparing Functionalized Compositions

In an aspect, provided herein is a method for preparing a functionalized composition. In some embodiments, the method comprises activating an analyte-binding composition comprising a plurality of functionalization sites at a surface. In some embodiments, the method comprises reacting the analyte-binding composition with a first functionalization agent. In some embodiments, the first functionalization agent is amine modified. In some embodiments, a first linker is interspaced between the amine and the first functionalization agent. In some embodiments, the method comprises linking a modified analyte to the first functionalization agent.

In another aspect, provided herein is a method for functionalizing a surface. In some embodiments, the method comprises providing a surface comprising a plurality of carboxylated functionalization sites. In some embodiments, the method comprises providing a functionalization agent comprising a primary amine and an analyte-binding region. In some embodiments, the method comprises covalently linking the functionalization agent to the plurality of carboxylated functional sites to obtain the functionalized surface. In some embodiments, the analyte-binding region may be configured to bind a target analyte.

### Analyte-Binding Compositions

In another aspect, provided herein is an analyte-binding composition. In some embodiments, the analyte-binding composition comprises a surface comprising a plurality of functionalization sites. In some embodiments, the analyte-binding composition comprises one or more functionalization agents covalently linked to at least two of the plurality of functionalization sites by one or more linking groups to yield a functionalized surface. In some embodiments, the covalently linked functionalization agents may be distributed across the functionalized surface within one standard deviation of a uniform linear spacing distance (dL) or within one standard deviation of a uniform radial spacing distance (dR).

In some embodiments, the one or more linking groups comprises a bond. In some embodiments, the one or more linking groups comprises an amide, an amine, an ester, or an azide. The one or more linking groups may comprise the amide. The one or more linking groups may comprise the amine. The one or more linking groups may comprise the azide.

In some embodiments, the one or more functionalization agents may be covalently linked to more than or equal to about: 3, 5, 10, or 20 of the plurality of functionalization sites. In some cases, the one or more functionalization agents are covalently linked to more than or equal to about: 1, 2, 3, 4, 5, 6, 7, 8, 9, 10, 15, 20, 25, 30, 35, 40, 45, or 50 of the plurality of functionalization sites. In some cases, the one or more functionalization agents are covalently linked to less than or equal to about: 1, 2, 3, 4, 5, 6, 7, 8, 9, 10, 15, 20, 25, 30, 35, 40, 45, or 50 of the plurality of functionalization sites.

In some embodiments, the analyte-binding composition is linked to a plurality of target analytes. In some embodiments, the analyte-binding composition is linked to about 10⁸ molecules or less per functionalized surface. In some cases, the analyte-binding composition is linked to less than or equal to about: 10⁵, 10⁶, 10⁷, 10⁸, 10⁹, 10¹⁰, or 10¹¹ molecules per functionalized surface. In some cases, the analyte-binding composition is linked to more than or equal to about: 10⁵, 10⁶, 10⁷, 10⁸, 10⁹, 10¹⁰, or 10¹¹ molecules per functionalized surface.

In some embodiments, a fluorescent tag may be covalently linked to the functionalized surface. In some embodiments, the fluorescent tag comprises fluorescein. In some embodiments, the covalently linked functionalization agents are distributed across the functionalized surface within one standard deviation of the uniform linear spacing distance (d_{L}). In some embodiments, the covalently linked functionalization agents are distributed across the functionalized surface within one standard deviation of a uniform radial spacing distance (d_{R}). In some embodiments, the one or more functionalization agents is covalently linked to the plurality of carboxylated functionalization sites via ring-strained copper-free click chemistry.

### Methods and Systems for Measuring Target Binding

In another aspect, disclosed herein is a method for measuring target binding. In some embodiments, the method comprises measuring target binding described herein may comprise providing a surface comprising a plurality of carboxylated functionalization sites. In some embodiments, the method comprises providing one or more functionalization agents comprising a primary amine, an analyte-binding region, and an optically active region. In some embodiments, the method comprises covalently linking the one or more functionalization agents to the plurality of carboxylated functional sites to obtain a functionalized surface. In some embodiments, the method comprises introducing the functionalized surface to a sample comprising a target analyte to promote binding between the analyte-binding region and the target analyte. In some embodiments, the method comprises optically measuring the binding between the functionalized surface and the target analyte.

In some cases, the method comprises optically measuring the binding. In some embodiments, optically measuring the binding may be performed using a member selected from the group consisting of a fluorescence microscope, a flow cytometer, a fluorescence spectrophotometer, a surface plasmon resonance detector, or an enzyme-linked immunosorbent assay (ELISA) detector.

In another aspect, the present disclosure provides a system for measuring target binding. In some embodiments, the system comprises an optical detector. In some embodiments, the system comprises any of the analyte-binding compositions described herein. In some embodiments, the system comprises a mass spectrometer. In some embodiments, the optical detector is selected from the group consisting of a fluorescence microscope, a flow cytometer, a fluorescence spectrophotometer, a surface plasmon resonance detector, or an enzyme-linked immunosorbent assay (ELISA) detector.

### Surfaces

In some embodiments, the surface or the functionalized surface comprises a bead. In some cases, the bead comprises a magnetic bead. In some embodiments, the bead comprises a carboxylated magnetic bead. In some cases, the bead comprises a Luminex bead. In some cases, the bead comprises a nanoparticle. In some cases, the nanoparticle comprises a carboxylated gold surface. In some cases, the nanoparticle comprises a carboxyl-functionalized magnetic nanoparticle. In some cases, the nanoparticle comprises a FluoSphere carboxylate-modified microsphere.

In some embodiments, the plurality of functionalization sites is uniformly distributed across the surface or the functionalized surface. In some embodiments, the plurality of functionalization sites is uniformly distributed across the surface or the functionalized surface within one standard deviation of a uniform linear spacing distance (d_{L}) or within one standard deviation of a uniform radial spacing distance (d_{R}). In some embodiments, d_{R} isless than or equal to about π/2. The uniform radial spacing distance (d_{R}) may be less than or equal to about π/4. In some cases, d_{R} is less than or equal to about: π/16, π/12, π/8, π/6, π/4, π/3, π/2, or π. In some cases, d_{R} is more than or equal to about: π/16, π/12, π/8, π/6, π/4, π/3, π/2, or π.

A diameter of the bead may be less than or equal to about 10 micrometers (μm). A diameter of the bead may be about 10 nanometers (nm), 20 nm, 50 nm, 100 nm, 200 nm, 500 nm, 1 μm, 2 μm, 3 μm, 4 μm, 5 μm, 6 μm, 7 μm, 8 μm, 9 μm, 10 μm, 11 μm, 12 μm, 13 μm, 14 μm, 15 μm, 16 μm, 17 μm, 18 μm, 19 μm, 20 μm, 21 μm, 22 μm, 23 μm, 24 μm, or 25 μm. In some cases, the diameter is more than or equal to about: 10 nm, 20 nm, 50 nm, 100 nm, 200 nm, 500 nm, 1 μm, 2 μm, 3 μm, 4 μm, 5 μm, 6 μm, 7 μm, 8 μm, 9 μm, 10 μm, 11 μm, 12 μm, 13 μm, 14 μm, 15 μm, 16 μm, 17 μm, 18 μm, 19 μm, 20 μm, 21 μm, 22 μm, 23 μm, 24 μm, or 25 μm. In some cases, the diameter is less than or equal to about: 10 nm, 20 nm, 50 nm, 100 nm, 200 nm, 500 nm, 1 μm, 2 μm, 3 μm, 4 μm, 5 μm, 6 μm, 7 μm, 8 μm, 9 μm, 10 μm, 11 μm, 12 μm, 13 μm, 14 μm, 15 μm, 16 μm, 17 μm, 18μ, 19 μm, 20μ, 21 μm, 22 μm, 23 μm, 24 μm, or 25μ m

In some embodiments, d_{L} is less than or equal to about: 5 μm, 4 μm, 3 μm, 2 μm, 1 μm, 100 nm, 10 nm, 1 nm, or 0.1 nm. In some embodiments, d_{L} is more than or equal to about: 5 μm, 4 μm, 3 μm, 2 μm, 1 μm, 100 nm, 10 nm, 1 nm, or 0.1 nm. In some embodiments, d_{L} is less than or equal to about: 5 μm, 4 μm, 3 μm, 2 μm, 1 μm, 100 nm, 10 nm, 1 nm, or 0.1 nm.

In some embodiments, d_{L} is configured for a modified analyte or analyte-binding region to link to the first functionalization agent at a first site, and link to the second functionalization agent at a second site. In some cases, d_{L} corresponds to a size, shape, or orientation of the first functionalization agent, the second functionalization agent, the first, second, or third linkers, the analyte-binding region, or a combination thereof. In some cases, the first functionalization agent, the second functionalization agent, the first, second, or third linkers, or the analyte-binding region may be determined based at least in part on d_{L}. For example, shorter linkers may be used for small values of d_{L}, while longer linker lengths may be preferable for larger values of d_{L}.

In some embodiments, the surface or the functionalized surface comprises a planar surface. In some cases, the planar surface comprises a carboxylate-functionalized surface plasmon resonance (SPR) surface. In some cases, the planar surface comprises a surface of a gold chip. In some cases, the surface of the gold chip is modified with one or more carboxyl active groups. In some cases, the planar surface comprises a Sartorius COOH1 sensor chip.

### Functionalization Agents

In some embodiments, the first functionalization agent comprises dibenzocyclooctyne (DBCO). In some cases, the DBCO is configured to couple with a modified analyte comprising an azide. In some embodiments, the first functionalization agent comprises an alkyne, a linear alkyne, an azide, a tetrazine, an alkene, a ring-strained alkyne, a terminal thiol, a thiol reactive agent, or a combination thereof. In some cases, alkene comprises trans-cyclooctene. In some cases, the ring-strained alkyne comprises DBCO. In some cases, the terminal thiol comprises cysteine. In some cases, the thiol reactive reagent comprises a maleimide, a haloacetyl, a pyridyl disulfide, a vinyl sulfone, an epoxide, or a combination thereof.

In some embodiments, the one or more functionalization agents may comprise an amine, an azide, or a cycloalkyne. The amine may comprise an amine modified cyclooctyne. The amine modified cyclooctyne, may be a primary amine modified dibenzocyclooctyne (DBCO).

In some cases, the linear alkyne is configured to couple with a modified analyte comprising an azide. In some cases, the azide is configured to couple with a modified analyte comprising a linear alkyne. In some cases, the tetrazine is configured to couple with a modified analyte comprising an alkene. In some cases, the alkene is configured to couple with a modified analyte comprising a tetrazine. In some cases, the ring-strained alkyne is configured to couple with a modified analyte comprising an azide. In some cases, the azide is configured to couple with a modified analyte comprising a ring-strained alkyne. In some cases, the terminal thiol is configured to couple with a modified analyte comprising a thiol reactive reagent. In some cases, the thiol reactive group is configured to couple with a modified analyte comprising a terminal thiol.

In some embodiments, a total length of the one or more functionalization agents may be configured to promote binding or interaction between the functionalized surface and a target analyte. In some embodiments, the target analyte may be a protein, a peptide, a nucleotide, an antibody, or an aptamer. In some cases, the target analyte comprises a small molecule, a peptide molecule, a nucleic acid molecule, a metal, an antibody, an antigen, a metabolite, a cell, or a combination thereof. In some embodiments, the target analyte comprises the antibody. In some embodiments, the target analyte comprises the peptide. In some embodiments, the target analyte comprises the protein

In some embodiments, the one or more functionalization agents comprise one or more antigen-binding sites. In some embodiments, more than or equal to about: 30%, 50%, 80%, or 95% of the one or more antigen-binding sites may be oriented toward an outer interface of the functionalized surface. In some cases, more than or equal to about: 10%, 20%, 30%, 40%, 50%, 60%, 70%, 75%, 80%, 85%, 90%, or 95% of the one or more antigen-binding sites are oriented toward the outer interface of the functionalized surface. In some cases, less than or equal to about: 10%, 20%, 30%, 40%, 50%, 60%, 70%, 75%, 80%, 85%, 90%, or 95% of the one or more antigen-binding sites are oriented toward the outer interface of the functionalized surface

In some embodiments, the one or more functionalization agents comprise a polyethylene glycol (PEG). In some embodiments, the one or more functionalization reagents may comprise a compound of the formula:

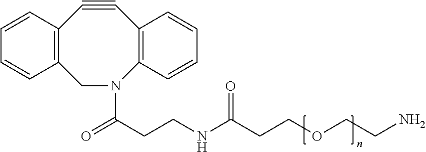

In some embodiments, n may be 1 to 30. In some embodiments, n may be 2, 4, 8, 12, 16, 24, or 28. In some embodiments, n may be 4. In some embodiments, n may be 8. In some embodiments, n may be 12. In some embodiments, n may be 23.

### Linkers

In some embodiments, the first linker comprises an alkyl, a polyglycerol, a polysarcosine, an amino acid spacer, a triazole, a carbamate, a carbonate, a zwitterionic spacer, or a combination thereof. In some cases, the polyglycerol comprises a branched hydrophilic polymer. In some cases, the polysarcosine comprises N-methylglycine. In some cases, the amino acid spacer comprises a glycine. In some cases, the amino acid spacer comprises more than or equal to about: 1, 2, 3, 4, 5, 10, 15, 20, 25, 30, 35, 40, 45, or 50 amino acids. In some cases, the amino acid spacer comprises less than or equal to about: 1, 2, 3, 4, 5, 10, 15, 20, 25, 30, 35, 40, 45, or 50 amino acids. In some cases, the zwitterionic space comprises sulfobetaine or carboxybetaine.

In some embodiments, the first linker comprises polyethylene glycol (PEG)-4. In some embodiments, the first linker comprises PEG-2, PEG-4, PEG-8, PEG-12, PEG-16, PEG-20, PEG-24, or PEG-28.

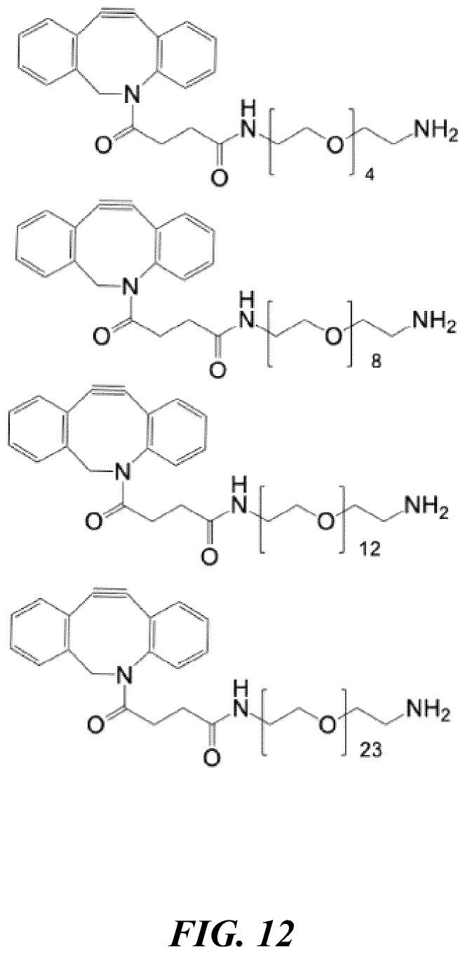

FIG. 12 illustrates examples of functionalization agents comprising polyethylene glycol (PEG) linkers of various lengths. As illustrated in FIG. 12, the first functionalization agent may comprise a DBCO group and an amine group. The first functionalization agent may comprise a first linker interspaced between the amine and the DBCO. The linker may comprise PEG. The linker may comprise PEG-4, PEG-8, PEG-12, PEG-23, or a combination thereof.

In some embodiments, the linking is performed using one or more click-chemistry reactions. In some cases, the linking is performed using 1-ethyl-3-(3-dimethylaminopropyl) carbodiimide (EDC) and/or N-hydroxysuccinimide (NHS). In some cases, the one or more click-chemistry reactions are configured to improve an efficiency, yield, or specificity of the linking. In some cases, the one or more click-chemistry reactions are configured to be performed under mild conditions.

### Modified Analytes and Linkers

In some embodiments, the modified analyte comprises an alkyne, a linear alkyne, an azide, a tetrazine, an alkene, a cycloalkyne, a terminal thiol, a thiol reactive agent, a maleimide, a haloacetyl, a pyridyl disulfide, a vinyl sulfone, an epoxide, or a combination thereof. In some cases, the modified analyte may comprise a complementary group to the first functionalization agent. For example, if the first functionalization may comprise a terminal thiol, the modified analyte may comprise the thiol reactive agent.

In some embodiments, the modified analyte comprises an azide modified analyte. In some embodiments, the azide in the azide modified analyte reacts with the DBCO of the first functionalization agent via a second linker. In some cases, the second linker comprises an alkyl, a polyglycerol, a polysarcosine, an amino acid spacer, a triazole, a carbamate, a carbonate, a zwitterionic spacer, or a combination thereof. In some cases, the second linker is the same as the first linker. In some cases, the second linker is different than the first linker. In some embodiments, the second linker comprises a polyethylene glycol (PEG). In some embodiments, the PEG is a PEG-N comprising PEG-1, PEG-2, PEG-3, PEG-4, PEG-5, PEG-6, PEG-7, PEG-8, PEG-9, or PEG-10. In some embodiments, N is more than or equal to about: 1, 2, 3, 4, 5, 6, 7, 8, 9, 10, 20, 30, 40, 50, 100, 200, 300, 400, or 500. In some embodiments, N is less than or equal to about: 1, 2, 3, 4, 5, 6, 7, 8, 9, 10, 20, 30, 40, 50, 100, 200, 300, 400, or 500.

In some embodiments, the method may comprise modifying the first linker, the second linker, or both. The modifying may comprise a chemical reaction. The modifying may comprise adding a group or removing a group from the first linker or the second linker. The modifying may comprise increasing or decreasing a length of the first linker or the second linker. The modifying may comprise linking the first linker or the second linker to the plurality of functionalization sites, the first functionalization agent, or the modified analyte. The modifying may comprise separating the first linker or the second linker from the plurality of functionalization sites, the first functionalization agent, or the modified analyte. In some embodiments, the modifying promotes binding of the first linker, the second linker, or both to a surface or a target analyte. In some embodiments, the modifying of the first linker, the second linker, or both, is performed at least in part to optimize a separation distance between the amine and the first functionalization agent.

In some embodiments, the first or the second modified linker comprises PEG-1, PEG-2, PEG-3, PEG-4, PEG-5, PEG-6, PEG-7, PEG-8, PEG-9, or PEG-10. In some embodiments, the modifying of the first linker, the second linker, or both, is used to direct an orientation of the first functionalization agent with respect to the plurality of functionalization sites. In some cases, the predicted orientation is determined based at least in part on one or more experimental assays testing one or more linkers. In some cases, the orientation of the first functionalization agent may be determined based on computational modeling of the plurality of functionalization sites, the first functionalization agent, the modified analyte, or a combination thereof. In some cases, the orientation of the first functionalization agent may be determined based at least in part on a density or spacing between the plurality of functionalization sites. In some cases, the orientation of the first functionalization agent may be determined based at least in part on a size, shape, or orientation of the analyte-binding region or target analyte.

### Target-Binding Moieties

In some embodiments, the modified analyte comprises a target-binding moiety. In some embodiments, the one or more functionalization agents may comprise an analyte-binding region. In some embodiments, the analyte-binding region comprises a target-binding moiety.

In some embodiments, the target binding moiety comprises a peptide. In some cases, the peptide comprises an antibody. In some cases, the peptide comprises an antigen recognition sequence. In some cases, the peptide comprises a fragment of a protein. In some cases, the peptide is configured to bind the target analyte.

In some embodiments, the peptide comprises less than or equal to about 100 amino acids. In some embodiments, the peptide comprises more than or equal to 10 amino acids. In some embodiments, the peptide comprises about 20 to 40 amino acids. In some embodiments, the peptide comprises more than or equal to 40 amino acids. In some cases, the peptide comprises more than or equal to about: 5, 10, 15, 20, 25, 30, 35, 40, 45, 50, 60, 7—, 80, 90, 100, 150, 200, 250, 300, 350, 400, 450, or 500 amino acids. In some cases, the peptide comprises less than or equal to about: 5, 10, 15, 20, 25, 30, 35, 40, 45, 50, 60, 70, 80, 90, 100, 150, 200, 250, 300, 350, 400, 450, or 500 amino acids.

In some embodiments, the target binding moiety comprises an aptamer. In some cases, the aptamer comprises a sequence of one or more nucleic acid bases. In some cases, the aptamer comprises a sequence of one or more DNA bases. In some cases, the aptamer comprises a sequence of one or more RNA bases. In some cases, the peptide is configured to bind the target analyte.

In some embodiments, the aptamer comprises more than or equal to 30 bases. In some embodiments, the aptamer comprises less than or equal to about 100 bases. In some embodiments, the aptamer comprises about 40 to about 100 bases. In some embodiments, the aptamer comprises about 50 bases. In some cases, the aptamer comprises more than or equal to about: 5, 10, 15, 20, 25, 30, 35, 40, 45, 50, 60, 7—, 80, 90, 100, 150, 200, 250, 300, 350, 400, 450, or 500 bases. In some cases, the aptamer comprises less than or equal to about: 5, 10, 15, 20, 25, 30, 35, 40, 45, 50, 60, 70, 80, 90, 100, 150, 200, 250, 300, 350, 400, 450, or 500 bases.

### Dual Display

In some embodiments, the method comprises reacting the modified analyte with a second functionalization agent. In some cases, the second functionalization agent comprises dibenzocyclooctyne (DBCO). In some cases, the DBCO is configured to couple with a modified analyte comprising an azide. In some embodiments, the first functionalization agent comprises an alkyne, a linear alkyne, an azide, a tetrazine, an alkene, a ring-strained alkyne, a terminal thiol, a thiol reactive agent, or a combination thereof. In some cases, alkene comprises trans-cyclooctene. In some cases, the ring-strained alkyne comprises DBCO. In some cases, the terminal thiol comprises cysteine. In some cases, the thiol reactive reagent comprises a maleimide, a haloacetyl, a pyridyl disulfide, a vinyl sulfone, an epoxide, or a combination thereof.

In some embodiments, the second functionalization agent comprises a same chemical structure as the first functionalization agent. In some embodiments, the second functionalization agent comprises a different chemical structure as the first functionalization agent. In some embodiments, the method comprises determining a predicted orientation of the modified analyte, based at least in part on the first functionalization agent or the second functionalization agent. In some embodiments, the method comprises selecting the first functionalization agent or the second functionalization agent from a plurality of potential functionalization agents, based at least in part on the predicted orientation.

In some cases, the predicted orientation is determined based at least in part on one or more experimental assays testing one or more combinations of first and second functionalization agents. In some cases, the predicted orientation may be determined based at least in part on computational modeling of the plurality of functionalization sites, the first functionalization agent, the modified analyte, or a combination thereof. In some cases, the predicted orientation may be determined may be determined based at least in part on a density or spacing between the plurality of functionalization sites. In some cases, the predicted orientation may be determined may be determined based at least in part on a size, shape, or orientation of the analyte-binding region or target analyte.

In some embodiments, the second functionalization agent is amine modified. In some embodiments, a third linker is interspaced between the amine and the second functionalization agent. In some cases, the third linker comprises an alkyl, a polyglycerol, a polysarcosine, an amino acid spacer, a triazole, a carbamate, a carbonate, a zwitterionic spacer, or a combination thereof. In some cases, the third linker is the same as the first linker or the second linker. In some cases, the third linker is different than the first linker or the second linker. In some embodiments, the third linker is longer than the first linker or the second linker. In some embodiments, the third linker is shorter than the first linker or the second linker.

In some embodiments, the method comprises determining a predicted orientation of the modified analyte, based at least in part on a length of the first linker, the second linker, the third linker, or a combination thereof. some embodiments, the method comprises selecting the length of the first linker, the second linker, the third linker, or a combination thereof from a plurality of potential lengths, based at least in part on the predicted orientation.

In some cases, the predicted orientation is determined based at least in part on one or more experimental assays testing one or more combinations of first and third linkers. In some cases, the predicted orientation may be determined based at least in part on computational modeling of the plurality of functionalization sites, the first functionalization agent, the modified analyte, the first linker, the second linker, the third linker, or a combination thereof. In some cases, the predicted orientation may be determined may be determined based at least in part on a density or spacing between the plurality of functionalization sites. In some cases, the predicted orientation may be determined may be determined based at least in part on a size, shape, or orientation of the analyte-binding region or target analyte.

In some embodiments, the third linker comprises a PEG-N comprising PEG-1, PEG-2, PEG-3, PEG-4, PEG-5, PEG-6, PEG-7, PEG-8, PEG-9, or PEG-10. In some embodiments, Nis more than or equal to about: 1, 2, 3, 4, 5, 6, 7, 8, 9, 10, 20, 30, 40, 50, 100, 200, 300, 400, or 500. In some embodiments, N is less than or equal to about: 1, 2, 3, 4, 5, 6, 7, 8, 9, 10, 20, 30, 40, 50, 100, 200, 300, 400, or 500.

In some embodiments, the method comprises linking the modified analyte to the second functionalization agent. In some cases, the linking may be performed using any of the methods disclosed herein (e.g., click chemistry, EDC/NHS, etc.) In some embodiments, the modified analyte is linked to the first functionalization agent via a C-terminal and linked to the second functionalization agent via an N-terminal. In some embodiments, the modified analyte is modified at the N-terminal or the C-terminal. In some cases, the modified analyte is modified at the N-terminal and the C-terminal. In some cases, the modified analyte is modified with an alkyne, a linear alkyne, an azide, a tetrazine, an alkene, a cycloalkyne, a terminal thiol, a thiol reactive agent, a maleimide, a haloacetyl, a pyridyl disulfide, a vinyl sulfone, an epoxide, or a combination thereof. In some cases, the modified analyte is modified at the N-terminal and the C-terminal using a same group. In some cases, the modified analyte is modified at the N-terminal and the C-terminal using different groups.

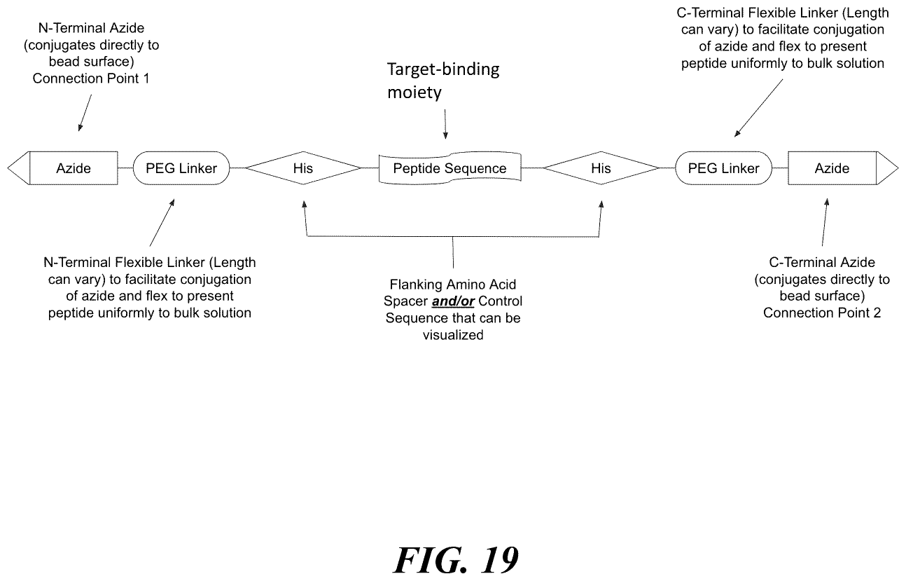

FIG. 19 illustrates an example of an analyte-binding region modified at two sites. As illustrated in FIG. 19, the analyte-binding region may comprise a target-binding moiety. The target-binding moiety may comprise a peptide sequence. The peptide sequence may be flanked by one or more tags. The one or more tags may comprise amino acid spacers, control tag sequences, or both. The one or more tags may be visualized to determine a signal provided by the methods disclosed herein. The one or more tags may be flanked by two linkers. The two linkers may comprise a N-terminal linker and a C-terminal linker. The length or chemical structure of the N-terminal linker and the C-terminal linker may vary, depending on the functionalization agent, the plurality of functionalization sites, the modified analyte, the analyte-binding region, or the target analyte. The N-terminal linker or the C-terminal linker may be modified with an azide. The azide may allow the analyte-binding region to link to two functionalization agents at the N-terminal and the C-terminal end.

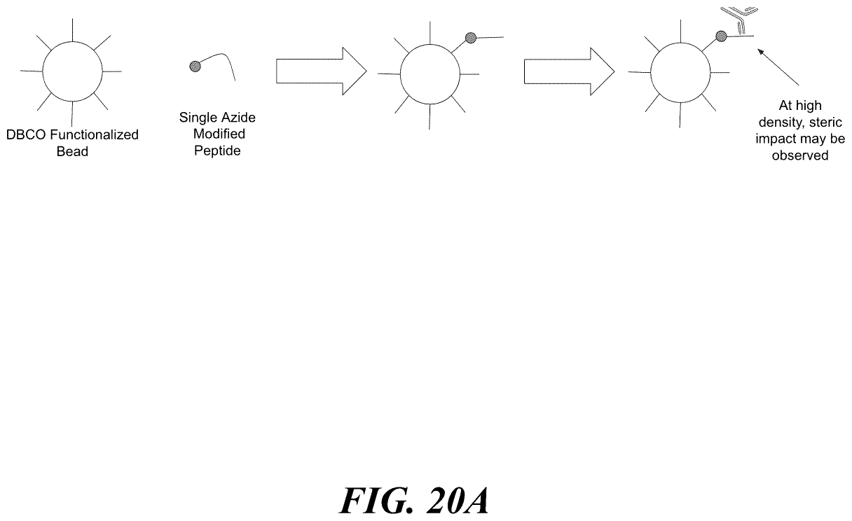

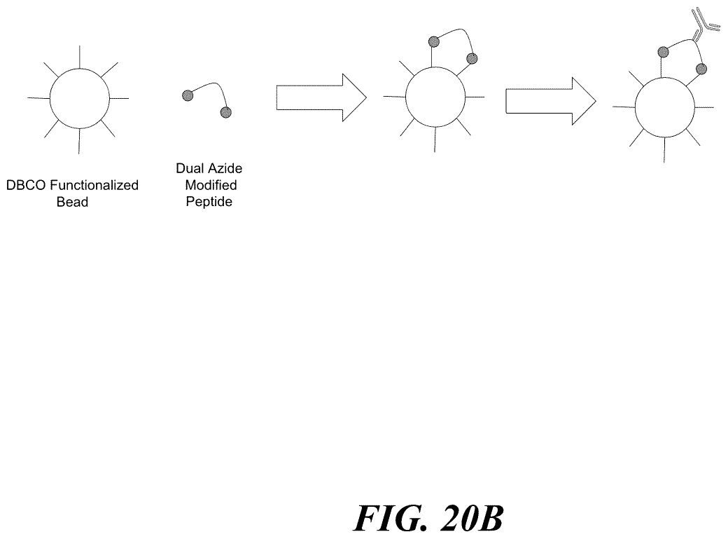

FIG. 20 illustrates examples of an analyte-binding region linked to one or two functionalization agents. As illustrated in FIG. 20A, an analyte-binding region may be modified at a single site, and may link to a functionalization agent at the modified site.

Alternatively, or in addition, an analyte-binding region may be modified at two or more sites and may link to two or more functionalization agents via the two or more modified sites, as illustrated in FIG. 20B. This may improve an orientation of the analyte-binding region or decrease a steric hindrance, depending on the analyte-binding region, the density of the plurality of functionalization sites, the target analyte, or a combination thereof.

In another aspect, the present disclosure provides a method for preparing a functionalized composition. In some embodiments, the method comprises activating an analyte-binding composition comprising a plurality of functionalization sites. In some embodiments, the method comprises reacting the analyte-binding composition with a first functionalization agent. In some embodiments, the first functionalization agent is amine modified. In some embodiments, a first linker is interspaced between the amine and the first functionalization agent. In some embodiments, the method comprises reacting the analyte-binding composition with a second functionalization agent. In some embodiments, the second functionalization agent is amine modified. In some embodiments, a second linker is interspaced between the amine and the second functionalization agent. In some embodiments, the method comprises coupling a modified analyte to the first functionalization agent at a first site and to the second functionalization agent at a second site.

In another aspect, the present disclosure provides an analyte-binding composition. In some embodiments, the analyte-binding composition comprises a surface comprising a plurality of functionalization sites. In some embodiments, the analyte-binding composition comprises a first functionalization agent linked via a first linker to a first functionalization site of the plurality of functionalization sites. In some embodiments, the analyte-binding composition comprises a second functionalization agent linked via a second linker to a second functionalization site of the plurality of functionalization sites. In some embodiments, the first functionalization site and the second functionalization site are separated by a uniform linear distance (d_{L}).

### Computer Systems

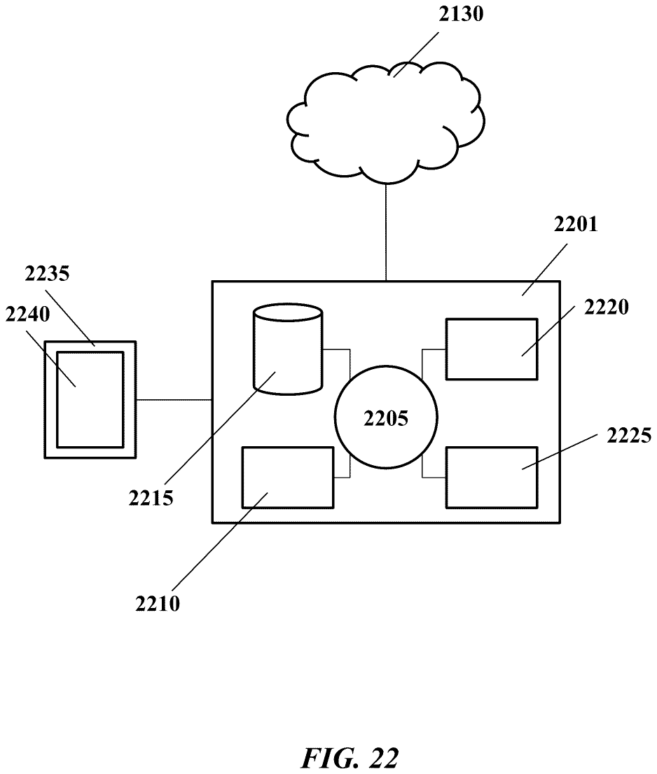

The present disclosure provides computer systems that may be programmed to implement methods of the disclosure. FIG. 22 shows an example of a computer system 2201 that is programmed or otherwise configured to perform analysis of peptides, proteins, and/or antibodies utilizing binding compositions. The computer system 2201 can regulate various aspects of analyte measurement and/or characterization of the present disclosure, such as, for example, optical or mass spectrometry measurements of analyte-binding compositions and/or complexes of analyte-binding compositions with analytes, as described herein. The computer system 2201 may be an electronic device of a user or a computer system that is remotely located with respect to the electronic device. The electronic device may be a mobile electronic device.

The computer system 2201 may include a central processing unit (CPU, also “processor” and “computer processor” herein) 2205, which may be a single core or multi core processor, or a plurality of processors for parallel processing. The computer system 2201 may also include memory or memory location 2210 (e.g., random-access memory, read-only memory, flash memory), electronic storage unit 2215 (e.g., hard disk), communication interface 2220 (e.g., network adapter) for communicating with one or more other systems, and peripheral devices 2225, such as cache, other memory, data storage and/or electronic display adapters. The memory 2210, storage unit 2215, interface 2220 and peripheral devices 2225 may be in communication with the CPU 2205 through a communication bus (solid lines), such as a motherboard. The storage unit 2215 may be a data storage unit (or data repository) for storing data. The computer system 2201 may be operatively coupled to a computer network (“network”) 2230 with the aid of the communication interface 2220. The network 2230 may be the Internet, an internet and/or extranet, or an intranet and/or extranet that is in communication with the Internet. The network 2230 in some cases may be a telecommunication and/or data network. The network 2230 can include one or more computer servers, which can enable distributed computing, such as cloud computing. The network 2230, in some cases with the aid of the computer system 2201, can implement a peer-to-peer network, which may enable devices coupled to the computer system 2201 to behave as a client or a server.

The CPU 2205 can execute a sequence of machine-readable instructions, which may be embodied in a program or software. The instructions may be stored in a memory location, such as the memory 2210. The instructions may be directed to the CPU 2205, which can subsequently program or otherwise configure the CPU 2205 to implement methods of the present disclosure. Examples of operations performed by the CPU 2205 can include fetch, decode, execute, and writeback.

The CPU 2205 may be part of a circuit, such as an integrated circuit. One or more other components of the system 2201 may be included in the circuit. In some cases, the circuit is an application specific integrated circuit (ASIC).

The storage unit 2215 can store files, such as drivers, libraries and saved programs. The storage unit 2215 can store user data, e.g., user preferences and user programs. The computer system 2201 in some cases can include one or more additional data storage units that are external to the computer system 2201, such as located on a remote server that is in communication with the computer system 2201 through an intranet or the Internet.

The computer system 2201 can communicate with one or more remote computer systems through the network 2230. For instance, the computer system 2201 can communicate with a remote computer system of a user. Examples of remote computer systems include personal computers (e.g., portable PC), slate or tablet PC's (e.g., Apple® iPad, Samsung® Galaxy Tab), telephones, Smart phones (e.g., Apple® iphone, Android-enabled device, Blackberry®), or personal digital assistants. The user can access the computer system 2201 via the network 2230.

Methods as described herein may be implemented by way of machine (e.g., computer processor) executable code stored on an electronic storage location of the computer system 2201, such as, for example, on the memory 2210 or electronic storage unit 2215. The machine executable or machine readable code may be provided in the form of software. During use, the code may be executed by the processor 2205. In some cases, the code may be retrieved from the storage unit 2215 and stored on the memory 2210 for ready access by the processor 2205. In some situations, the electronic storage unit 2215 may be precluded, and machine-executable instructions are stored on memory 2210.

The code may be pre-compiled and configured for use with a machine having a processer adapted to execute the code, or may be compiled during runtime. The code may be supplied in a programming language that may be selected to enable the code to execute in a pre-compiled or as-compiled fashion.

Aspects of the systems and methods provided herein, such as the computer system 2201, may be embodied in programming. Various aspects of the technology may be thought of as “products” or “articles of manufacture” typically in the form of machine (or processor) executable code and/or associated data that is carried on or embodied in a type of machine readable medium. Machine-executable code may be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 2201 can include or be in communication with an electronic display 2235 that comprises a user interface (UI) 2240 for providing, for example, configurable analyte measurement parameters. Examples of UI's include, without limitation, a graphical user interface (GUI) and web-based user interface.

Methods and systems of the present disclosure may be implemented by way of one or more algorithms. An algorithm may be implemented by way of software upon execution by the central processing unit 2205. The algorithm can, for example, be configured to facilitate the performance of any of the methods described herein.

### EXAMPLES

### Example 1: Preparation of DBCO Functionalized Bead Compositions

Carboxylated Luminex Magnetic beads were activated with EDC/NHS which made them reactive with primary amines.

Once the Luminex beads were activated, they were subsequently reacted with primary amine modified DBCO, a cyclooctyne that reacts with azides though ring-strained copper-free click chemistry. A 4-unit polyethylene glycol (PEG) polymer repeat for the spacer (also referred to as PEG4) was used. Highly specific “click chemistry” was used for the chemical conjugation between the DBCO-modified Luminex beads and the analyte of interest. An assay utilizing the PEG4 composition is illustrated in FIG. 6. A composition using an 8-unit PEG spacer was synthesized using an analogous process and the same assay as FIG. 6 was repeated. The results for the 8-unit PEG spacer are shown in FIG. 7.

### Example 2: Comprehensive Component Evaluation of the Analyte-Binding Compositions Describe Herein

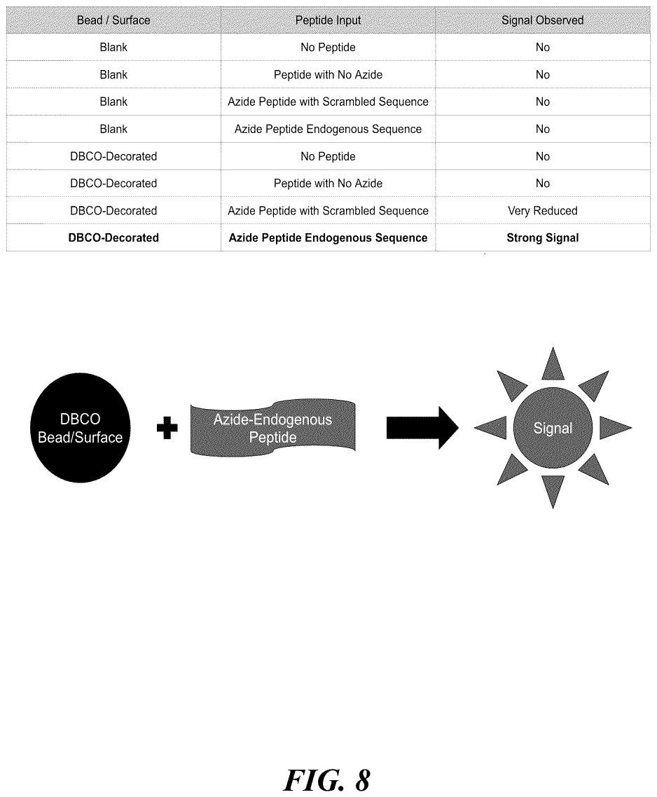

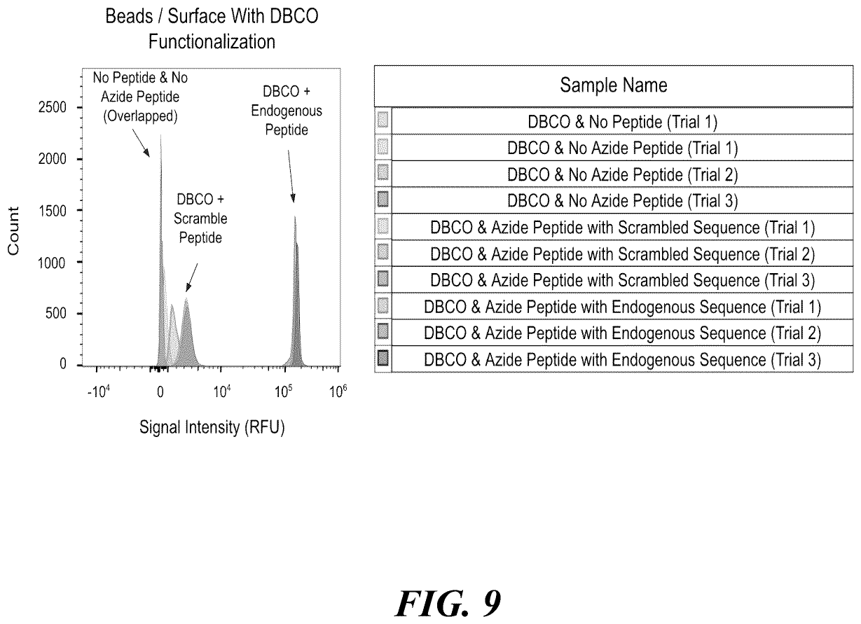

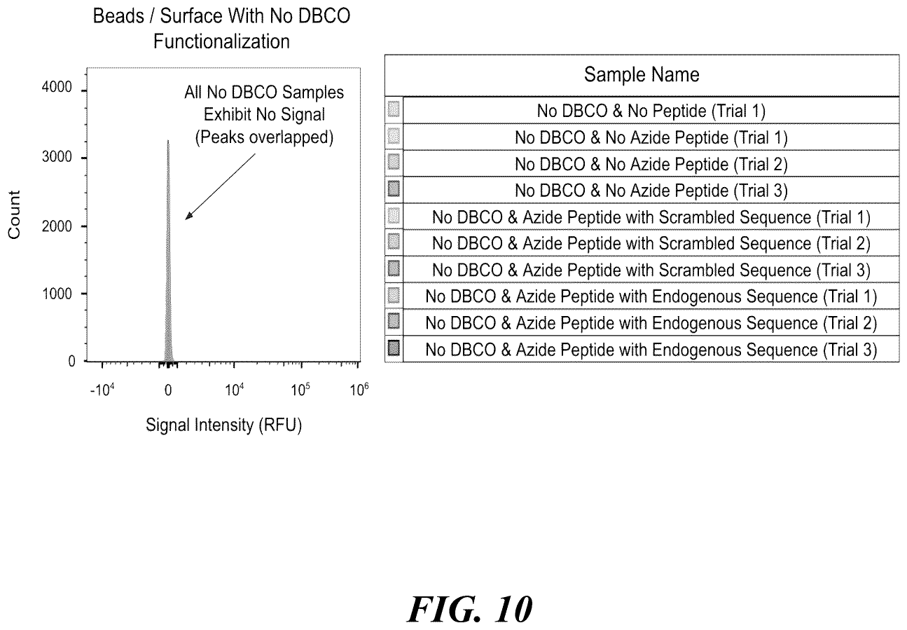

A comprehensive component knockout evaluation in an analyte-binding assay was performed. The description and results are provided in FIG. 8 which shows that the assay signal was eliminated in all assay conditions where any single aspect of the analyte-binding workflow described herein was not present. This is further highlighted in FIG. 9, which provides ten distinct analyte-bind assay conditions and the respective assay signals indicative of target analyte binding for each. These results show that all assay components, including properly chemically tagged peptides with the proper sequence, are required to provide a signal above the threshold demonstrating binding of the detection antibody to a target analyte. Furthermore, FIG. 10 illustrates that beads without proper functionalization as described herein used in a binding assay resulted in the elimination of all assay signal.

### Example 3: Human Plasma Sample Analysis by the Analyte-Binding Composition Described Herein

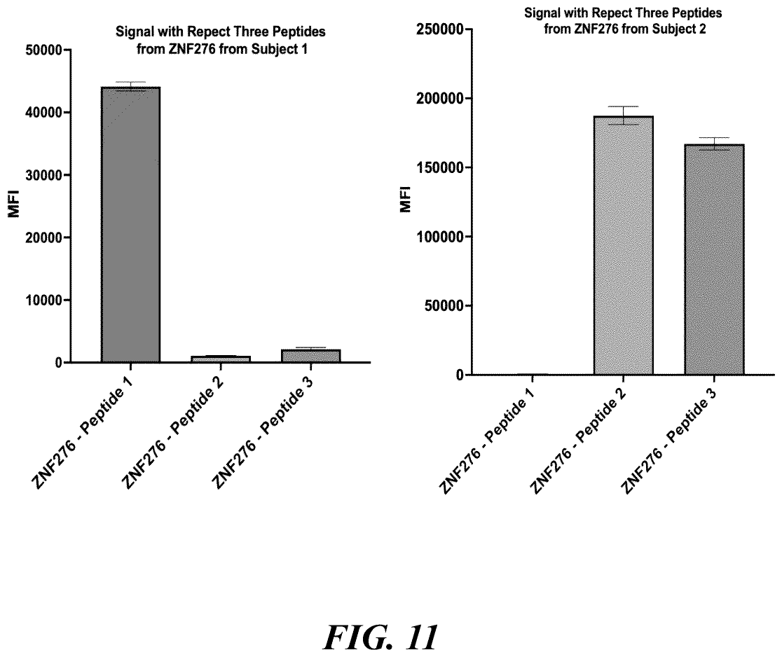

Demonstration of utility in human plasma samples was assessed. Beads were functionalized with DBCO-PEG4-Amine; click chemistry was then utilized to specifically conjugate azide-tagged antigens of interest to beads in a uniform fashion. In FIG. 11, three different sets of DBCO-modified beads were conjugated with three different and distinct azide modified peptides of interest. Human blood samples were obtained from two different individuals and processed though standard methods. Plasma samples were then diluted 200-fold in aqueous buffer. Diluted plasma samples were then incubated separately with the three different peptide coated beads populations. Nonspecific interactions were then removed through comprehensive washing. Specific peptide-analyte interactions were then detected using fluorescent-based measurement approaches. In subject 1, peptide 1 coated beads were the only peptide coated beads that produced signal while Peptide 2 and Peptide 3 coated beads produced no signal. Complementary to this observation, in subject 2, Peptide 2 and Peptide 3 coated beads both produced significant signal while Peptide 1 coated beads produced no signal. These results highlight the measurement of specific biological diversity found between human subjects.

### Example 4: Analysis of Analyte-Binding Fragments Comprising Protein Fragments

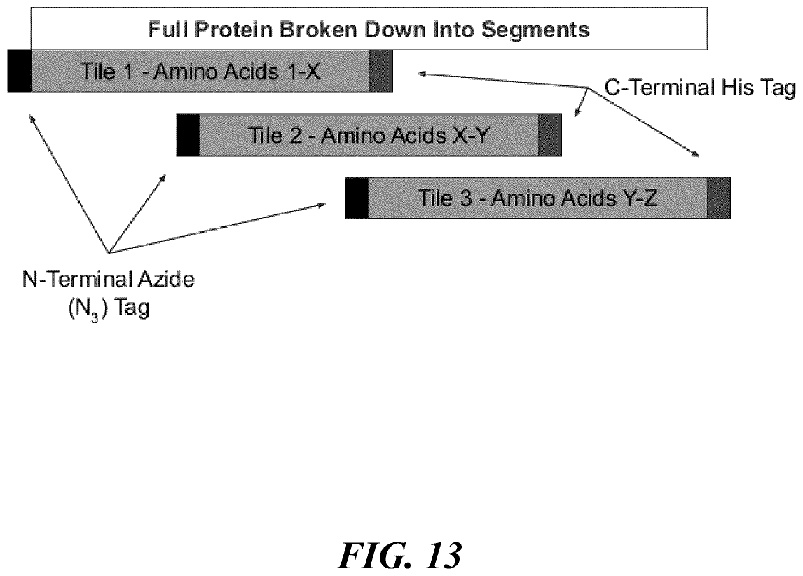

FIG. 13 illustrates examples of target-binding moieties comprising peptides. As illustrated in FIG. 13, the target-binding moiety may comprise a fragment of a protein. The fragment may be flanked by a tag comprising an azide modification, which may allow the modified analyte to be linked to the first functionalization agent.

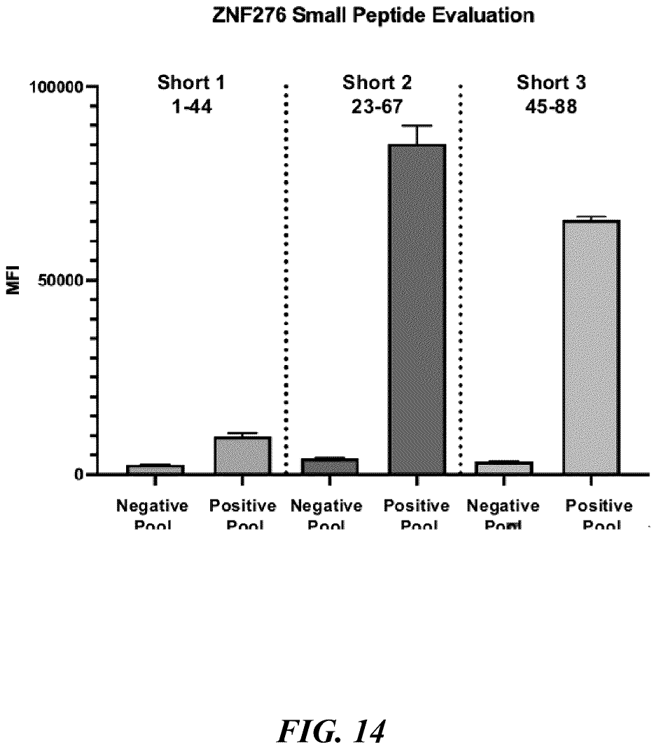

FIG. 14 illustrates an assay of analytical performance of example analyte-binding compositions having target-binding moieties comprising peptides. A signal may be determined based on assaying multiple fragments of a protein (e.g., zinc finger protein 276) in one or more samples. The one or more samples may be divided into a subset of positive samples (samples comprising the target analyte) and a subset of negative samples (samples not comprising the target analyte). As illustrated in FIG. 14, the subset of positive samples have high signal for the second fragment and the third fragment of the protein, and relatively lower signal for the first fragment. This may allow the identification of the target binding region of the protein. Using a target-binding moiety comprising a fragment of the protein may additionally increase signal, resolution, or specificity of the methods disclosed herein.

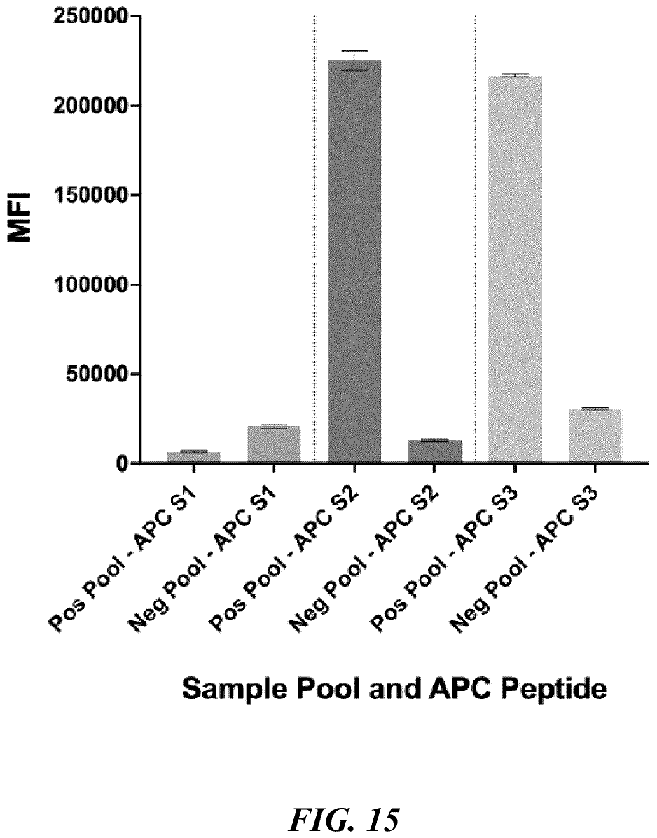

FIG. 15 illustrates an assay of analytical performance of example analyte-binding compositions having target-binding moieties comprising peptides. A signal may be determined based on assaying multiple fragments of a protein (e.g., Adenomatous polyposis coli) in one or more positive samples and one or more negative samples. As illustrated in FIG. 15, the one or more positive samples have high signal for the second fragment and the third fragment of the protein, and relatively lower signal for the first fragment, which may be indicative of a target binding region of the protein.

### Example 5: Evaluation of Click Chemistry Vs. EDC/NHS for Preparing Analyte-Binding Compositions

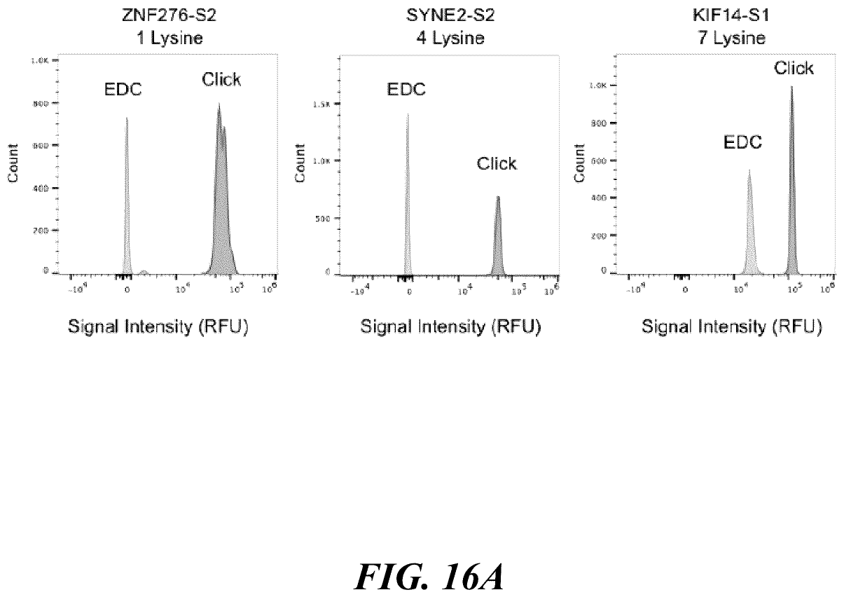

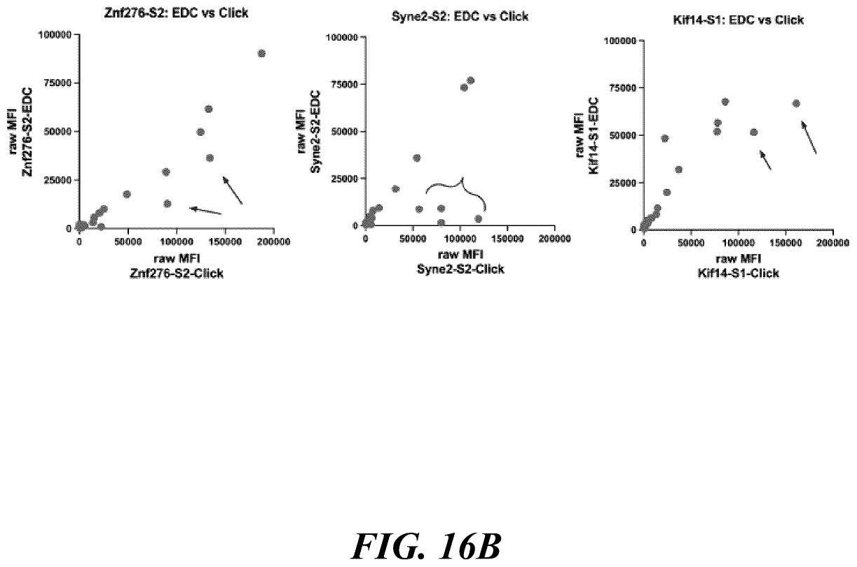

FIG. 16A illustrates histograms of the signal intensity (x-axis) obtained using click-chemistry and EDC/NHS when assaying analytes comprising different lysine contents. As illustrated in FIG. 16A, click-chemistry provided higher signal compared to EDC/NHS. EDC/NHS comprises conjugation via lysine amino acid residues, so EDC/NHS can provide a relatively high signal when lysine content is higher. However, as illustrated in FIG. 16A, click-chemistry provided higher signal than EDC/NHS, even in the analyte comprising the highest lysine content.

FIG. 16B illustrates the analytical performance of click-chemistry (x-axis) against the performance of EDC/NHS (y-axis) for plasma samples. As illustrated in FIG. 16B, all samples had higher signal using click-chemistry compared to EDC/NHS, and a subset of the samples have particularly high signal when using click-chemistry, (e.g., are closer to the x-axis).

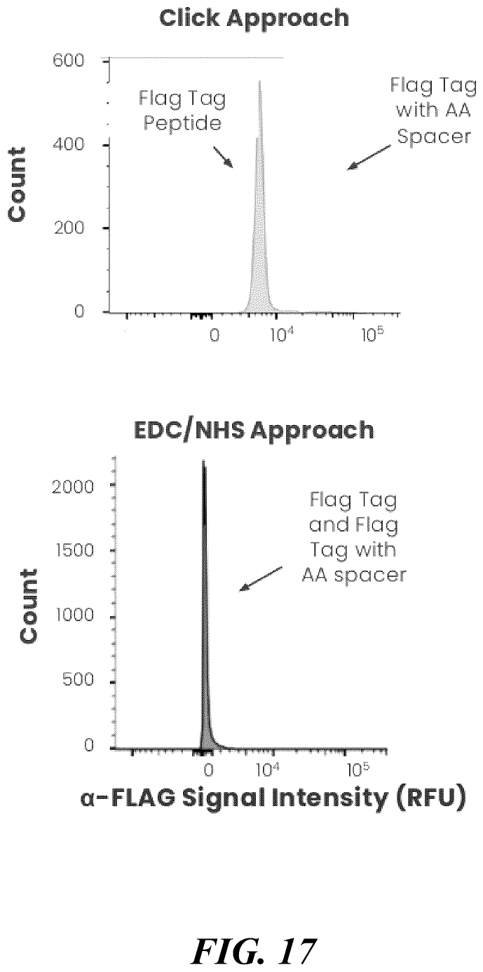

FIG. 17 illustrates an assay of analytical performance of an example analyte-binding composition. To evaluate an impact of analyte size on detection, a Flag tag peptide (8 amino acids) and a size normalized Flag peptide comprising an amino acid spacer (total 24 amino acids) were assayed. As shown in FIG. 17, the click-chemistry approach provided signal for both the Flag tag peptide and the size-normalized Flag peptide, while no signal was provided for either analyte using EDC/NHS. Therefore, the click-chemistry linking and analyte-binding compositions provided herein provide improved performance when assaying small analytes, compared to other methods for functionalizing a surface.

### Example 6: Evaluation of Analyte-Binding Composition Linker Lengths

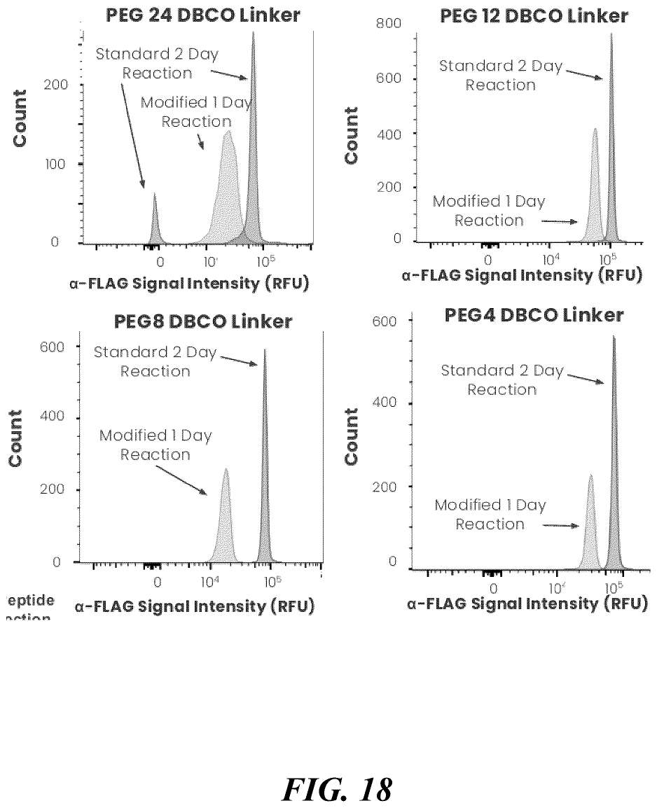

FIG. 18 illustrates an assay of analytical performance of example analyte-binding compositions comprising various linker lengths. As illustrated in FIG. 18, 4 linker lengths were evaluated under 2 different reaction conditions. The standard 2-day reaction included an overnight incubation between the DBCO-modified beads and the modified analyte, under shaking conditions (1750 RPM) at 25 C in the dark. The modified 1-day reaction was used to determine if the reaction time could be decreased to 4 hours with shaking (1750 RPM) at 25 C. As shown in FIG. 18, fluorescent signal is still observed and is higher than EDC/NHS, but the standard 2 day reaction provides higher signal. To evaluate an impact of linker length, 4 different PEG linkers (PEG-24, PEG-12, PEG-8, and PEG-4) were used with small peptides (22 amino acids). As shown in FIG. 18, PEG-12 provided the highest signal intensity, although PEG-4 provided higher signal intensity for other analytes. This may indicate that different linker lengths may be more suitable for different analytes. Also, PEG-24 resulted in a heterogenous population of beads, resulting in 2 peaks.

### Example 7: Comparison of Single-Linked and Dual-Linked Analyte-Binding Regions

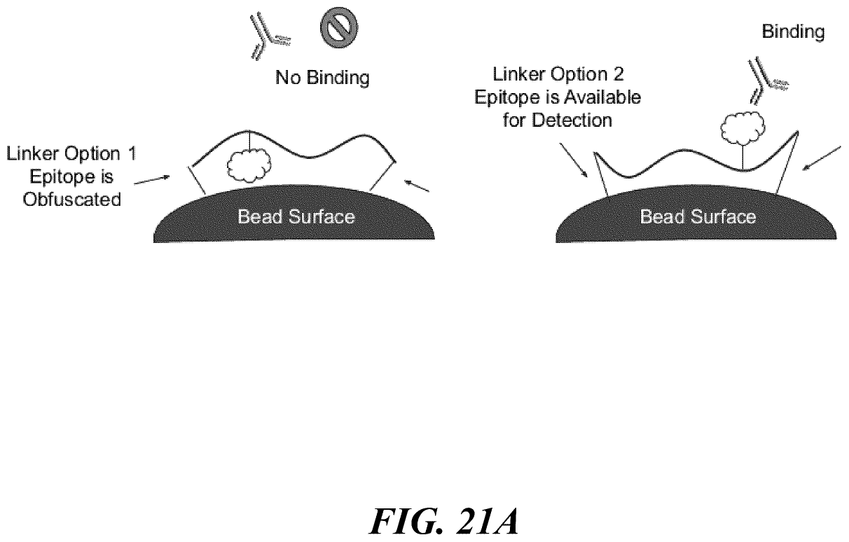

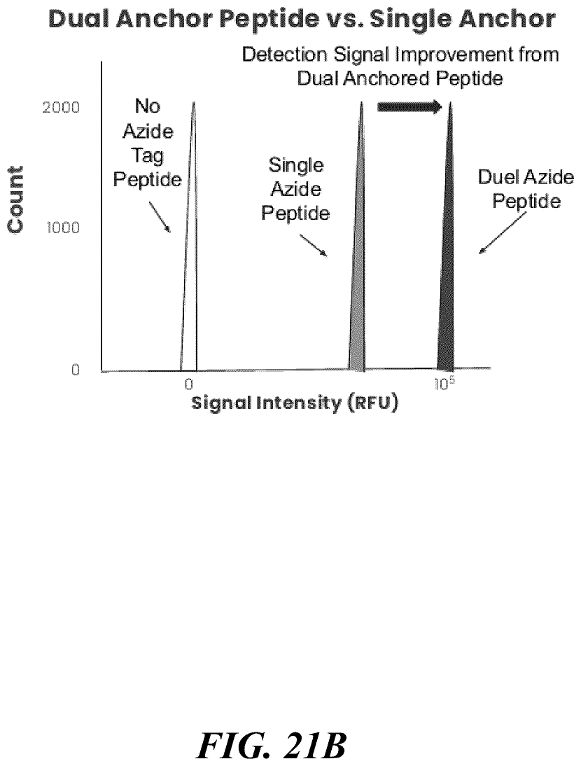

FIG. 21A illustrates an example of an orientation of an analyte-binding region linked to two functionalization agents. As illustrated in FIG. 21A, the orientation of the analyte-binding region of the analyte-binding composition may affect a binding of the target analyte. For example, the analyte-binding region may be positioned to face away from the target analyte, depending on the density or spacing of the plurality of functionalization sites, the length of the first, second, or third linkers, the analyte-binding region, or the target analyte. Changing the density or spacing of the plurality of functionalization sites, the length of the first, second, or third linkers, the analyte-binding region, or the target analyte may change the orientation of the analyte-binding region to face the target analyte, thereby improving binding.

FIG. 21B illustrates an analytical performance of analyte-binding region linked to one functionalization agent, as compared to two functionalization agents. As illustrated in FIG. 21B, the analyte-binding region linked to two functionalization agents may be positioned to facilitate binding of the target analyte, thereby improving the signal detection.

While preferred embodiments of the present inventive concepts have been shown and described herein, it will be obvious to those skilled in the art that such embodiments are provided by way of example only. It is not intended that the inventive concepts be limited by the specific examples provided within the specification. While the inventive concepts have been described with reference to the aforementioned specification, the descriptions and illustrations of the embodiments herein are not meant to be construed in a limiting sense. Numerous variations, changes, and substitutions will now occur to those skilled in the art without departing from the inventive concepts. Furthermore, it shall be understood that all aspects of the inventive concepts are not limited to the specific depictions, configurations or relative proportions set forth herein which depend upon a variety of conditions and variables. It should be understood that various alternatives to the embodiments of the inventive concepts described herein may be employed in practicing the inventive concepts. It is therefore contemplated that the inventive concepts shall also cover any such alternatives, modifications, variations, or equivalents. It is intended that the following claims define the scope of the inventive concepts and that methods and structures within the scope of these claims and their equivalents be covered thereby.

1.-243. (canceled)

244. A method of preparing a functionalized composition, the method comprising:
(a) activating an analyte-binding composition comprising a plurality of functionalization sites;
(b) reacting the analyte-binding composition with a first functionalization agent, wherein the first functionalization agent is amine modified, and wherein a first linker is interspaced between the amine and the first functionalization agent;
(c) reacting the analyte-binding composition with a second functionalization agent, wherein the second functionalization agent is amine modified, and wherein a second linker is interspaced between the amine and the second functionalization agent; and
(d) coupling a modified analyte to the first functionalization agent at a first site and to the second functionalization agent at a second site.

245. The method of claim 244, wherein the first functionalization agent comprises dibenzocyclooctyne (DBCO).

246. The method of claim 244, wherein the first functionalization agent comprises an alkyne, a linear alkyne, an azide, a tetrazine, an alkene, a ring-strained alkyne, a terminal thiol, a thiol reactive agent, or a combination thereof.

247. The method of claim 244, wherein the first linker comprises an alkyl, a polyglycerol, a polysarcosine, an amino acid spacer, a triazole, a carbamate, a carbonate, a zwitterionic spacer, or a combination thereof.

248. The method of claim 244, wherein the first linker comprises PEG-2, PEG-4, PEG-8, PEG-12, PEG-16, PEG-20, PEG-24, or PEG-28.

249. The method of claim 244, wherein the first linker comprises PEG-4.

250. The method of claim 244, wherein the modified analyte comprises an alkyne, a linear alkyne, an azide, a tetrazine, an alkene, a cycloalkyne, a terminal thiol, a thiol reactive agent, a maleimide haloacetyl, a pyridyl disulfide, a vinyl sulfone, an epoxide, or a combination thereof.

251. The method of claim 250, wherein the modified analyte comprises an azide modified analyte.

252. The method of claim 251, wherein the azide in the azide modified analyte reacts with the DBCO of the first functionalization agent via a second linker.

253. The method of claim 252, wherein the second linker comprises a polyethylene glycol (PEG).

254. The method of claim 253, wherein the PEG is a PEG-N comprising PEG-1, PEG-2, PEG-3, PEG-4, PEG-5, PEG-6, PEG-7, PEG-8, PEG-9, or PEG-10.

255. The method of claim 254, wherein N is more than or equal to about: 1, 2, 3, 4, 5, 6, 7, 8, 9, 10, 20, 30, 40, 50, 100, 200, 300, 400, or 500.

256. The method of claim 254, wherein N is less than or equal to about: 1, 2, 3, 4, 5, 6, 7, 8, 9, 10, 20, 30, 40, 50, 100, 200, 300, 400, or 500.

257. The method of claim 252, further comprising modifying the first linker, the second linker, or both.

258. The method of claim 257, wherein the modifying promotes binding of the first linker, the second linker, or both to the surface or a target analyte.

259. The method of claim 257, wherein the modifying of the first linker, the second linker, or both, is performed at least in part to optimize a separation distance between the amine and the first functionalization agent.

260. The method of claim 259, wherein the first functionalization agent comprises DBCO.

261. The method of claim 257, wherein the first or the second modified linker comprises PEG-1, PEG-2, PEG-3, PEG-4, PEG-5, PEG-6, PEG-7, PEG-8, PEG-9, or PEG-10.

262. The method of claim 257, wherein the modifying of the first linker, the second linker, or both, is used to direct an orientation of the first functionalization agent with respect to the plurality of functionalization sites.

263. The method of claim 257, wherein the first functionalization agent is dispersed more uniformly across the plurality of functionalization sites as compared to a functionalized composition prepared using a linker that is unmodified.

264. The method of claim 244, wherein the modified analyte comprises a target binding moiety.

265. The method of claim 264, wherein the target binding moiety comprises a peptide.

266. The method of claim 265, wherein the peptide comprises less than or equal to about 100 amino acids.

267. The method of claim 265, wherein the peptide comprises more than or equal to 10 amino acids.

268. The method of claim 265, wherein the peptide comprises about 20 to 40 amino acids.

269. The method of claim 265, wherein the peptide comprises more than or equal to 40 amino acids.

270. The method of claim 264, wherein the target binding moiety comprises an aptamer.

271. The method of claim 270, wherein the aptamer comprises more than or equal to 30 bases.

272. The method of claim 270, wherein the aptamer comprises less than or equal to about 100 bases.

273. The method of claim 270, wherein the aptamer comprises about 40 to about 100 bases.

274. The method of claim 270, wherein the aptamer comprises about 50 bases.
